(19) United States
(12) Patent Application Publication
(10) Pub. No.: US 2024/0331807 A1
(43) Pub. Date: Oct. 3, 2024

(54) ARTIFICIAL INTELLIGENCE-GUIDED MOLECULAR SCREENING FOR COORDINATION FRAMEWORK COMPOUNDS

(71) Applicants: GE Infrastructure Technology LLC, Greenville, SC (US); University of California Berkeley, Berkeley, CA (US); University of Chicago, Chicago, IL (US)

(72) Inventors: Bojun Feng, Niskayuna, NY (US); Saumil Praphool Chheda, Berkeley, CA (US); Gautam Stroscio, Livermore, CA (US); Wooseok Jeong, Daejeon (KR); Nikita Hanikel, Seattle, WA (US); Jason F. Nichols, Niskayuna, NY (US); Laura Gagliardi, Chicago, IL (US); Omar Mwannes Yaghi, Berkeley, CA (US); David Roger Moore, Rexford, NY (US)

(21) Appl. No.: 18/593,296

(22) Filed: Mar. 1, 2024

Related U.S. Application Data
(60) Provisional application No. 63/488,307, filed on Mar. 3, 2023.

(51) Int. Cl. G16C 10/00; C07F 5/06; G06N 3/0455; G16C 20/30; G16C 20/70

(52) U.S. Cl. CPC G16C 10/00; C07F 5/069; G06N 3/0455; G16C 20/30; G16C 20/70

(57) ABSTRACT

Described herein are methods and systems for proposing coordination framework compounds, such as crystalline porous materials, crystalline open frameworks, reticular chemistry compounds, metal-organic framework (MOF) compounds, covalent organic framework (COF) compounds, zeolitic imidazolate framework (ZIF) compounds, and combinations thereof. Also described herein are coordination framework compounds produced by same and sorbent systems including the coordination framework compounds. The methods and systems described herein combine machine learning and chemistry to propose chemically valid and performance improved coordination framework compounds that meet different goals of material discovery.

### RELATED APPLICATIONS

This application claims priority to U.S. Provisional Patent Application No. 63/488,307, filed on Mar. 3, 2023, the contents of which are hereby incorporated by reference in their entirety.

### GOVERNMENT SUPPORT CLAUSE

This invention was made with government support under HR001121C0020 awarded by the Defense Advanced Research Projects Agency (DARPA). The government has certain rights in the invention.

### BACKGROUND

The field of the disclosure relates generally to methods and systems for proposing coordination framework compounds, such as crystalline porous materials, crystalline open frameworks, reticular chemistry compounds, metal-organic framework (MOF) compounds, covalent organic framework (COF) compounds, zeolitic imidazolate framework (ZIF) compounds, and combinations thereof. The field of the disclosure also relates to coordination framework compounds produced by same.

Coordination framework compounds, such as MOF, ZIF, and COF compounds, are useful for a wide variety of purposes. For example, they may be particularly useful in carbon capture sorbent systems or in methane capture sorbent systems, such as for use in post-combustion and direct air capture of carbon dioxide (CO₂). As another example, they are particularly useful in atmospheric water extraction (AWE), wherein water is generated remotely and on-demand. However, carbon capture and AWE processes each require constant and iterative improvements on materials.

Existing iterative trial-and-error methods of coordination framework compound discovery based on chemical knowledge require substantial investments and efforts to realize innovative and effective materials. Although pure machine learning (ML) methods can aid in discovery, they suffer from a lack of deep chemistry insights. For example, while the model developed by Xie et al. (“Crystal Diffusion Variational Autoencoder for Periodic Material Generation.” arXiv preprint arXiv:2110.06197 (2021)) advances machine learning capabilities for application to small unit-cell compounds (e.g., small crystals), it lacks chemical knowledge and guidance. This model was also only applied to small unit-cell compounds and not demonstrated for larger structures like MOF compounds.

Due to the deficiencies in existing methods, it would be desirable to develop a process that combines the strengths of machine learning-based and chemical knowledge-based methods in order to more effectively propose and discover coordination framework compounds.

### BRIEF DESCRIPTION

In one aspect, provided herein is a method of proposing a coordination framework compound. In the exemplary embodiment, the method is performed using a coordination framework compound proposal (CFCP) computing device that includes a processor coupled to a memory device. The exemplary method comprises: generating, using a machine learning model of the CFCP computing device, an initial set of coordination framework compounds; subjecting at least one of the coordination framework compounds of the initial set of coordination framework compounds to a review of at least one chemical property; and generating, using the CFCP computing device, a preliminary set of coordination framework compounds with the machine learning model based on the initial set of coordination framework compounds and the review of the at least one chemical property of at least one of the coordination framework compounds of the initial set of coordination framework compounds. The method also comprises proposing, using the CFCP computing device, the coordination framework compound.

In another aspect, provided herein is a coordination framework compound proposal (CFCP) computing device that comprises: a memory; and a processor communicatively coupled to the memory, wherein the processor is programmed to: generate an initial set of coordination framework compounds with a machine learning model; and to subject at least one of the coordination framework compounds of the initial set of coordination framework compounds to a review of at least one chemical property. The processor is further programmed to generate a preliminary set of coordination framework compounds with the machine learning model based on the initial set of coordination framework compounds and the review of the at least one chemical property of at least one of the coordination framework compounds of the initial set of coordination framework compounds; and to propose the coordination framework compound.

In still another aspect, provided herein is a non-transitory computer-readable storage medium having computer-executable instructions embodied thereon, wherein when executed by a coordination framework compound proposal (CFCP) computing device including at least one processor in communication with a memory, the computer-readable instructions cause the CFCP computing device to: generate an initial set of coordination framework compounds with a machine learning model; and to subject at least one of the coordination framework compounds of the initial set of coordination framework compounds to a review of at least one chemical property. The computer-readable instructions also cause the CFCP computing device to generate a preliminary set of coordination framework compounds with the machine learning model based on the initial set of coordination framework compounds and the review of the at least one chemical property of at least one of the coordination framework compounds of the initial set of coordination framework compounds; and to propose the coordination framework compound.

In still another aspect, provided herein is a coordination framework compound including: a plurality of secondary building units (SBUs), a plurality of linkers forming linkages between the plurality of SBUs, and a plurality of pores formed in interstices between the linkages. The coordination framework compound includes at least one of: at least two chemically different linkers, at least two geometrically different pores, and at least two linkages between two SBUs of the plurality of SBUs.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1A is an exemplary method flow chart in accordance with the present disclosure;

FIG. 1B is an exemplary method flow chart in accordance with the present disclosure;

FIG. 2 is an another exemplary method flow chart in accordance with the present disclosure;

FIG. 3 is a further exemplary method flow chart in accordance with the present disclosure;

FIG. 4 is yet another exemplary method flow chart in accordance with the present disclosure;

FIG. 5 is a block diagram of an exemplary computer system in accordance with the present disclosure;

FIG. 6 is an exemplary configuration of a server system, such as the computer system of FIG. 1A, in accordance with the present disclosure;

FIG. 7 is an exemplary configuration of a client system shown in FIG. 1A, in accordance with the present disclosure;

FIG. 8 is an exemplary method in accordance with the present disclosure;

FIG. 9 is an exemplary coordination framework compound obtained from a ML model in accordance with the present disclosure; and

FIG. 10 includes exemplary molecules used in replacement linkers in coordination framework compounds in accordance with the present disclosure.

FIG. 11 depicts performance between simulated comparative coordination framework compounds and coordination framework compounds in accordance with the present disclosure.

FIG. 12A depicts a first view of a coordination framework compound including a straight SBU topology in accordance with the present disclosure.

FIG. 12B depicts a second view of a coordination framework compound including a straight SBU topology in accordance with the present disclosure.

FIG. 13A depicts a first view of a coordination framework compound including a curly SBU topology in accordance with the present disclosure.

FIG. 13B depicts a second view of a coordination framework compound including a curly SBU topology in accordance with the present disclosure.

FIG. 14A depicts a first view of a coordination framework compound including an SBU topology that is neither straight nor curly in accordance with the present disclosure.

FIG. 14B depicts a second view of a coordination framework compound including an SBU topology that is neither straight nor curly in accordance with the present disclosure.

FIG. 15A depicts a first view of a coordination framework compound including a bridging arm linker structure in accordance with the present disclosure.

FIG. 15B depicts a second view of a coordination framework compound including a bridging arm linker structure in accordance with the present disclosure.

FIG. 16 depicts a coordination framework compound including a changed pore shape as a result of a mixture of different linkers in accordance with the present disclosure.

Unless otherwise indicated, the drawings provided herein are meant to illustrate features of embodiments of the disclosure. These features are believed to be applicable in a wide variety of systems, including one or more embodiments of the disclosure. As such, the drawings are not meant to include all conventional features known by those of ordinary skill in the art to be required for the practice of the embodiments disclosed herein.

### DETAILED DESCRIPTION

The embodiments described herein overcome at least some of the disadvantages of known methods of proposing coordination framework compounds. The present embodiments combine machine learning and chemistry to propose chemically valid and performance improved coordination framework compounds that meet different goals of material discovery. The machine learning provides material structure imagination and generation and the chemistry guidance suggests modifications and improvements to machine learning-generated materials.

As used herein, a coordination framework compound includes a node, which is a point for structural connectivity and gives rise to secondary building units (SBUs), one or more linkers, and a topology that defines how SBUs and linkers are connected together. Varying any of these aspects, either individually or in combination, results in a new and distinct coordination framework compound. The methods described herein are applicable to the discovery of any crystal structure.

Examples of coordination framework compounds include metal organic framework (MOF) compounds, covalent organic framework (COF) compounds, zeolitic imidazolate framework (ZIF) compounds, crystalline porous materials, crystalline open frameworks, reticular chemistry compounds, and combinations thereof. MOF compounds contain strong bonds between a metal atom and charged ligands and can include, but are not limited to only including, one or more types of metal atoms in the SBU and linker compositions, while COF compounds have strong covalent bonds between light elements (e.g., B, C, N, O, Si, P) and include, but are not limited to only including, one or more types of organic structures as the SBU. ZIFs are a subclass of MOF compounds that are composed of tetrahedrally-coordinated transition metal ions (e.g. Fe, Co, Cu, Zn) connected by imidazolate linkers that are topologically isomorphic with zeolites.

In general, coordination framework compounds are not limited to only having one SBU or one linker. Rather, they can have more complicated structures consisting of multiple types of nodes and linkers. In other words, the components of coordination framework compounds may be combined in a variety of ways. For example, the coordination framework compounds may include two linkers and one metal node.

The exemplary embodiments described herein include a method of proposing a coordination framework compound, wherein the method is performed using a coordination framework compound proposal (CFCP) computing device that includes a processor coupled to a memory device. The method includes: generating, using a machine learning model of the CFCP computing device, an initial set of coordination framework compounds; subjecting at least one of the coordination framework compounds of the initial set of coordination framework compounds to a review of at least one chemical property; generating, using the CFCP computing device, a preliminary set of coordination framework compounds with the machine learning model based on the initial set of coordination framework compounds and the review of the at least one chemical property of at least one of the coordination framework compounds of the initial set of coordination framework compounds; and proposing, using the CFCP computing device, the coordination framework compound.

The method may further include one or more additional process steps suitable to facilitate the method described herein. In some embodiments, the method also includes subjecting at least one of the coordination framework compounds of the preliminary set of coordination framework compounds to a review of at least one chemical property. In some embodiments, the method further includes validating at least one of the coordination framework compounds of the preliminary set of coordination framework compounds. In some embodiments, the method further includes performing at least one iteration of a sequence including: generating a further preliminary set of coordination framework compounds with the machine learning model based on at least one generated set of coordination framework compounds and a review of at least one chemical property of at least one of the coordination framework compounds of at least one generated set of coordination framework compounds; optionally subjecting at least one of the coordination framework compounds of the further preliminary set of coordination framework compounds to a review of at least one chemical property; and optionally validating at least one of the coordination framework compounds of the further preliminary set of coordination framework compounds.

The exemplified embodiments also include a coordination framework compound proposal (CFCP) computing device including a memory and a processor communicatively coupled to the memory, wherein the processor is programmed to carry out the method embodiments described herein.

The processor may be further programmed to perform one or more additional steps suitable to facilitate the method described herein. In some embodiments, the processor is further programmed to subject at least one of the coordination framework compounds of the preliminary set of coordination framework compounds to a review of at least one chemical property. In some embodiments, the processor is further programmed to validate at least one of the coordination framework compounds of the preliminary set of coordination framework compounds. In some embodiments, the processor is further programmed to perform at least one iteration of a sequence including: generating a further preliminary set of coordination framework compounds with the machine learning model based on at least one generated set of coordination framework compounds and a review of at least one chemical property of at least one of the coordination framework compounds of at least one generated set of coordination framework compounds; optionally subjecting at least one of the coordination framework compounds of the further preliminary set of coordination framework compounds to a review of at least one chemical property; and optionally validating at least one of the coordination framework compounds of the further preliminary set of coordination framework compounds.

The exemplary embodiments also include a non-transitory computer-readable storage medium having computer-executable instructions embodied thereon, wherein when executed by a coordination framework compound proposal (CFCP) computing device including at least one processor in communication with a memory. The computer-readable instructions cause the coordination framework compound proposal computing device to carry out the method embodiments described herein.

The computer-readable instructions may include one or more additional steps suitable to facilitate the method described herein. In some embodiments, the computer-readable instructions further cause the CFCP computing device to subject at least one of the coordination framework compounds of the preliminary set of coordination framework compounds to a review of at least one chemical property. In some embodiments, the computer-readable instructions further cause the CFCP computing device to validate at least one of the coordination framework compounds of the preliminary set of coordination framework compounds. In some embodiments, the computer-readable instructions further cause the CFCP computing device to perform at least one iteration of a sequence including: generating a further preliminary set of coordination framework compounds with the machine learning model based on at least one generated set of coordination framework compounds and a review of at least one chemical property of at least one of the coordination framework compounds of at least one generated set of coordination framework compounds; optionally subjecting at least one of the coordination framework compounds of the further preliminary set of coordination framework compounds to a review of at least one chemical property; and optionally validating at least one of the coordination framework compounds of the further preliminary set of coordination framework compounds.

Generally, any method according to the present disclosure may be implemented using a coordination framework compound proposal (CFCP) computing device. As used herein, a coordination framework compound proposal computing device includes any suitable computing device known in the art that facilitates the method described herein. Suitable computing devices may include, but are not limited to only including, computers, desktop computers, handheld computers, or smartphones.

Generally, the coordination framework compound may be any suitable coordination framework compound realized or proposed through the exemplary methods described herein. In some embodiments, the coordination framework compound is a coordination framework compound of the initial set of coordination framework compounds, a coordination framework compound of the preliminary set of coordination framework compounds, or a coordination framework compound of the further preliminary set of coordination framework compounds. In other words, the coordination framework compound may be a hypothesis coordination framework compound, a coordination framework compound realized by chemical insights into the hypothesis coordination framework compound, or coordination framework compound realized by iterative analysis of coordination framework compounds.

In some embodiments, the machine learning model is trained with existing coordination framework compounds, new coordination framework compounds, and combinations thereof. In some embodiments, the machine learning model is trained with existing coordination framework compounds.

Generally, the machine learning model may use any suitable technique that facilitates the exemplary methods described herein. In some embodiments, the machine learning model uses at least one of the following techniques: latent space, inverse search, variational autoencoder (VAE), crystal diffusion variational autoencoder (CDVAE), inverse search of VAE latent space, graph neural network (GNN), neural network, optimization, and combinations thereof. In some embodiments, the machine learning model is programmed to learn via artificial intelligence a latent space configured to reconstruct coordination framework compound crystal structures and accurately predict associated target properties.

In some embodiments, the machine learning model is programmed to learn via VAE (e.g. CDVAE) which maps coordination framework compound structures (e.g. the crystal structure of a MOF) to one point in a latent space, and then latent space reconstructs this point from the latent space to the original coordination framework compound structure. In this case, any point in the latent space corresponds to its correct crystal structure, and searching in the latent space may be performed to proposed new coordination framework compound structures. In addition, since each coordination framework compound will have certain target chemical properties, such as water isotherms, this latent space may also be mapped to accurately predict target chemical properties for every coordination framework compound. Therefore, each point in the latent space will correspond to its correct coordination framework compound as well as its target chemical properties. Inverse search and target properties optimization techniques may be performed to find new coordination framework compound with more desirable target chemical properties. This VAE approach may be combined with a GNN technique that maps crystal structures to machine-readable graphs that can be read and trained by a machine learning model.

In some embodiments, the at least one chemical property is selected from one or more of the following: adsorbate uptake capacity, adsorbate uptake kinetics, adsorbate gravimetric productivity, adsorbate volumetric productivity, adsorbate isotherms and isobars, pore size, pore volume, heat of adsorption, isosteric heat of adsorption, chemical stability, thermal stability, mechanical stability, synthetic feasibility, zeta potential, surface energy, hydrophobicity, hydrophilicity, chemical performance, chemical modifications for improvement, and combinations thereof.

In some embodiments, validating at least one of the coordination framework compounds includes experimentally validating the structure and/or properties of the at least one of the coordination framework compounds.

In some embodiments, experimentally validating the structure and/or properties of the at least one of the coordination framework compounds includes experimental validation with a technique selected from one or more of the following: powder x-ray diffraction (PXRD), single crystal X-ray diffraction, solid state nuclear magnetic resonance (SS-NMR), digestion NMR, and combinations thereof.

In some embodiments, experimentally validating the structure and/or properties of the at least one of the coordination framework compounds includes relaxing (e.g., electronically relaxing and/or optimizing) the structure using first-principles calculations to ensure that the coordination framework compound structure is stable. Then, pore volume and pore size of the coordination framework compound are calculated. Water stability and hydrophilicity are analyzed to validate whether the compound has the potential to absorb more water molecules. After this, computational water isotherms are calculated for the proposed coordination framework compound by Gibbs Ensemble Monte Carlo (GEMC) to estimate the compound's water adsorption ability. The proposed coordination framework compound is then validated by synthesis experiments and experimentally measured chemical properties, such as water isotherms.

In some embodiments, the machine learning model generates the preliminary coordination framework compound based on at least one of the following inputs: crystal structures of existing coordination framework compounds, target properties, target chemical properties, sorption isotherms, moisture sorption isotherms, pore volume, pore size, water stability, hydrophilicity, and combinations thereof.

In some embodiments, the review of at least one chemical property is performed by a machine, a human, or a combination thereof. In some embodiments, the review of at least one chemical property is performed by a human. In some embodiments, the review of at least one chemical property is performed using the CFCP computing device.

Generally, several challenges exist for ML applied to coordination framework compounds. These challenges include the invertibility of representations for periodic crystal structures as ML input, the invariance for crystal structures, and the large unit cell and number of atoms.

An exemplary periodic structure may be represented as M=(A, X, L), wherein N is the number of atoms; A∈A^{N}, atomic species; X∈R^{N×3}, atomic positions; L∈R^{3×3}, periodic lattice; c∈R^{|A|}, composition. Graphically, the periodic structure may be represented with an atom as a node and a bond as an edge.

Regarding invariance for materials, permutation invariance includes exchanging indices of any pair of atoms. Translation invariance includes translating X by an arbitrary vector. Rotation invariance includes rotating X and L together by an arbitrary rotation matrix. Periodic invariance includes infinite ways of unit cell with different shape and size.

ML training may be achieved in the following way. First, a periodic GNN encoder encodes M to z. Second, a property predictor predicts c, L and N of M and predicts target properties, such as water isotherms or pore volume (PV), from z. Third, a periodic GNN decoder denoises \tilde{M}, conditioned on z. Fourth, \tilde{M} is obtained by adding different levels of noise to X and A.

Material optimization may be achieved in the following way. First, start from existing structures M (e.g. MOF-303), and encode M to z. Second, optimize PV with respect to z, find z′ that gives desirable target properties, such as desirable water isotherms or larger PV. Third, use z′ to predict c, L and N. Fourth, randomly initialize an initial Mo. Fifth, decode M₀ to update a optimized material M′ that gives desirable target properties, such as desirable water isotherms or larger PV.

In some embodiments, a variational autoencoder-based ML model, such as a Crystal Diffusion Variational Autoencoder (CDVAE), is used to generate goal-oriented novel coordination framework compounds. In some preferred embodiments, a CDVAE is used to generate goal-oriented novel MOFs after being trained only on existing coordination framework compounds.

In some embodiments, a code base is used to perform deconstruction, modification, and/or reconstruction for ML-generated coordination framework compounds. Generally, the code base is compatible with any coordination framework compound. In some embodiments, the code base is selected from the group consisting of a ToBaCCo-based code base, a MOFid-based code base, a molfunc-based code base, and combinations thereof. In some embodiments, a ToBaCCo-based code base is used to perform deconstruction-modification-reconstruction for ML-generated coordination framework compounds with rod-like secondary building units (SBUs).

In many embodiments, the proposed coordination framework compound may be used according to any suitable purpose known in the art. In some embodiments, the proposed coordination framework compound is used in a sorbent system. In some embodiments, the proposed coordination framework compound is used in a carbon capture sorbent system. In some embodiments, the proposed coordination framework compound is used in a moisture sorbent system. In some embodiments, the proposed coordination framework compound is used for capturing a gas. In some embodiments, the proposed coordination framework compound is used for post-combustion capturing of CO₂ and/or direct air capturing of CO₂. In some embodiments, the proposed coordination framework compound is used for atmospheric water extraction.

In many embodiments, the proposed coordination framework compound comprises: a plurality of secondary building units (SBUs); a plurality of linkers forming linkages between the plurality of SBUs; and a plurality of pores formed in interstices between the linkages. The coordination framework compound comprises at least one of: at least two chemically different linkers; at least two geometrically different pores; and at least two linkages between two SBUs of the plurality of SBUs.

In some embodiments, the coordination framework compound comprises at least three linkages between two SBUs of the plurality of SBUs. In some embodiments, the coordination framework compound comprises at least four linkages between two SBUs of the plurality of SBUs. In some embodiments, the coordination framework compound comprises at least five linkages between two SBUs of the plurality of SBUs. In some embodiments, the coordination framework compound comprises at least six linkages between two SBUs of the plurality of SBUs.

In some embodiments, the coordination framework compound is selected from the group consisting of metal organic framework (MOF) compounds, covalent organic framework (COF) compounds, zeolitic imidazolate framework (ZIF) compounds, crystalline porous materials, crystalline open frameworks, reticular chemistry compounds, and combinations thereof.

In some embodiments, at least one SBU of the plurality of SBUs comprises a node comprising an atom selected from the group consisting of:
metal atoms, Al, or Mg;
B, C, N, O, Si, or P;
transition metal atoms, Fe, Co, Cu, or Zn; and
combinations thereof.

In some embodiments, at least one SBU of the plurality of SBUs comprises a coordination structure selected from the group consisting of polyhedral, tetrahedral, octahedral, cubic, dodecahedral, and combinations thereof.

In some embodiments, the coordination framework compound is planarly symmetrical. In these embodiments, the coordination framework compound that is planarly symmetrical may be MIL-53 type.

In some embodiments, the coordination framework compound is not planarly symmetrical. In these embodiments, the coordination framework compound that is not planarly symmetrical may be non-MIL-53 type.

In some embodiments, the at least two chemically different linkers comprise at least two linkers of different lengths.

In some embodiments, the at least two geometrically different pores differ by a geometric property selected from the group consisting of size, shape, and combinations thereof. In some embodiments, the geometric properties of pores may be altered by altering one or more of an atom of the node, the coordination structure of at least one SBU, the symmetry of the coordination framework compound, and the length of at least one linker. These various alterations and combinations thereof result in different shapes of SBUs, different connections between SBUs, and different pore geometries.

In some embodiments, the plurality of linkers comprises a chain of repeating linkers. In these embodiments, the repeating linkers may include a single repeating linker or a combination of different linkers in a random configuration or in a patterned configuration (e.g., repeating blocks of linkers). The chain of repeating linkers may include a small number of repeating linkers (e.g., less than 10) or a large number of repeating linkers (e.g., more than 1000).

In some embodiments, the plurality of linkers comprises a linker comprising at least one substituted or unsubstituted aryl ring, at least one substituted or unsubstituted fused aryl ring, at least two substituted or unsubstituted bonded aryl rings, or a combination thereof.

In some embodiments, the plurality of linkers comprises a linker selected from the group consisting of:

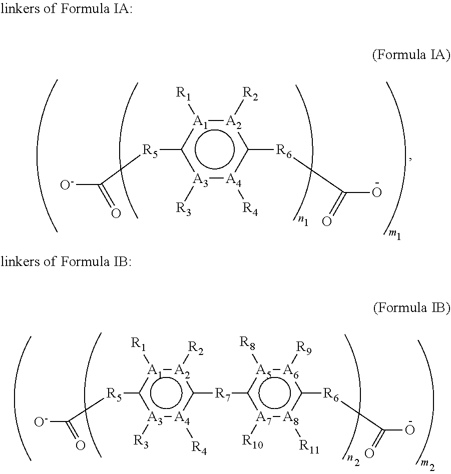

wherein:
n₁, m₁, n₂, and m₂ are each individually selected from the group consisting of 1, 2, 3, 4, 5, 6, 7, 8, 9, 10, integers no more than 100, integers no more than 1000, integers no more than 10000, integers no more than 100000, and integers no more than 1000000;
R₁, R₂, R₃, R₄, R₈, R₉, R₁₀, and R₁₁ are each individually selected from the group consisting of H, NH₂, OH, and SH;
R₅ and R₆ are each individually selected from the group consisting of direct bonds, R₁₂NHR₁₃, R₁₂OR₁₃, R₁₂SR₁₃, C₁-C₆ alkyl optionally substituted with at least one substituent selected from the group consisting of NH₂, OH, and SH, C₁-C₆ alkylene optionally substituted with at least one substituent selected from the group consisting of NH₂, OH, and SH, and combinations thereof;
R₇ is selected from the group consisting of direct bonds, ring fusions, NH, O, S, and C₁-C₆ alkyl;
R₁₂ and R₁₃ are each individually selected from the group consisting of direct bonds, NH, O, S, and C₁-C₆ alkyl; and
A₁, A₂, A₃, A₄, A₅, A₆, A₇, and A₈ are each individually selected from the group consisting of C, N, O, and S;

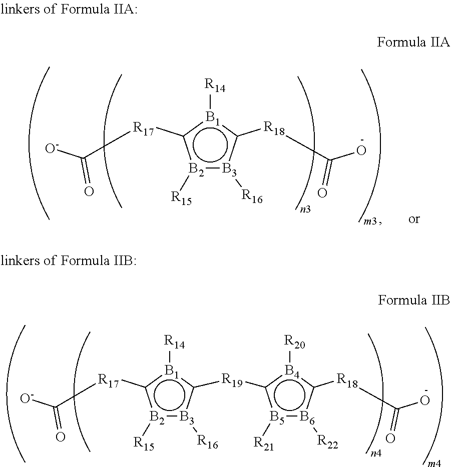

wherein:
n₃, m₃, n₄, and m₄ are each individually selected from the group consisting of 1, 2, 3, 4, 5, 6, 7, 8, 9, 10, integers no more than 100, integers no more than 1000, integers no more than 10000, integers no more than 100000, and integers no more than 1000000;
R₁₄, R₁₅, R₁₆, R₂₀, R₂₁, and R₂₂ are each individually selected from the group consisting of H, NH₂, OH, and SH;
R₁₇ and R₁₈ are each individually selected from the group consisting of direct bonds, R₂₃NHR₂₄, R₂₃OR₂₄, R₂₃SR₂₄, C₁-C₆ alkyl optionally substituted with at least one substituent selected from the group consisting of NH₂, OH, and SH, C₁-C₆ alkylene optionally substituted with at least one substituent selected from the group consisting of NH₂, OH, and SH, and combinations thereof;
R₁₉ is selected from the group consisting of direct bonds, ring fusions, NH, O, S, and C₁-C₆ alkyl;
R₂₃ and R₂₄ are each individually selected from the group consisting of direct bonds, NH, O, S, and C₁-C₆ alkyl; and
B₁, B₂, B₃, B₄, B₅, and B₆ are each individually selected from the group consisting of C, N, O, and S; and
combinations thereof.

In some embodiments, the plurality of linkers comprises a linker selected from the group consisting of:

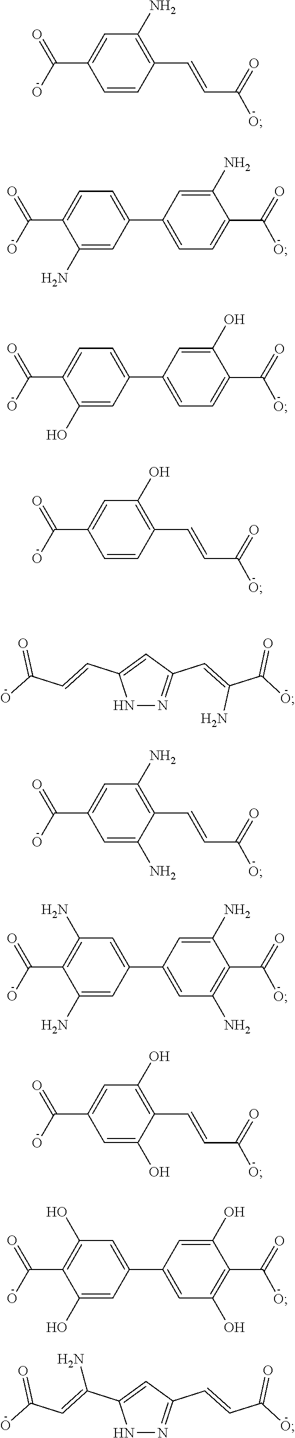

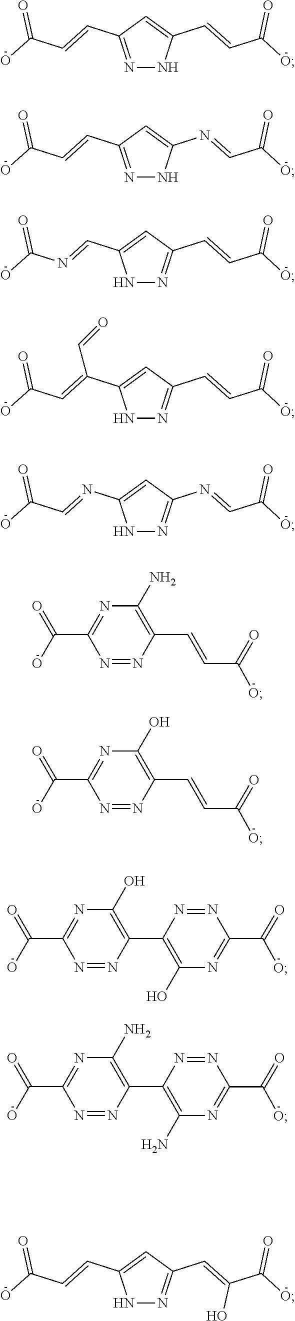

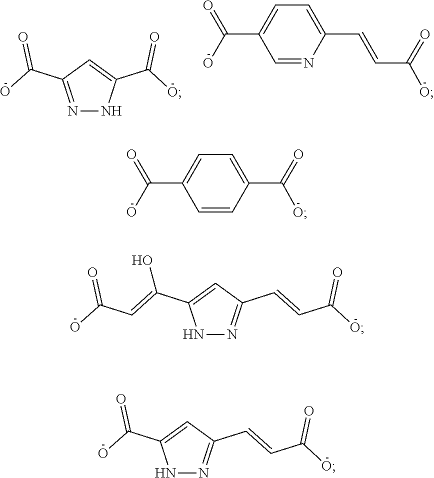

and combinations thereof.

In some embodiments, the plurality of linkers comprises a linker selected from the group consisting of:

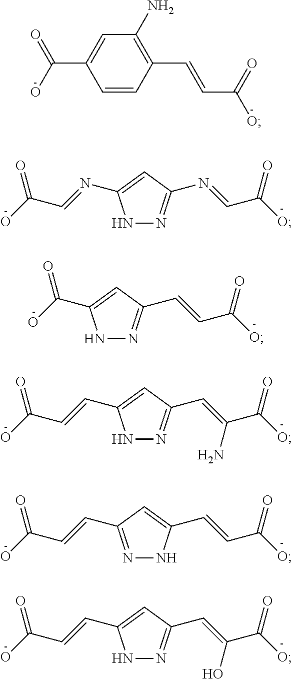

and combinations thereof.

In some embodiments, a sorbent system includes the coordination framework compound.

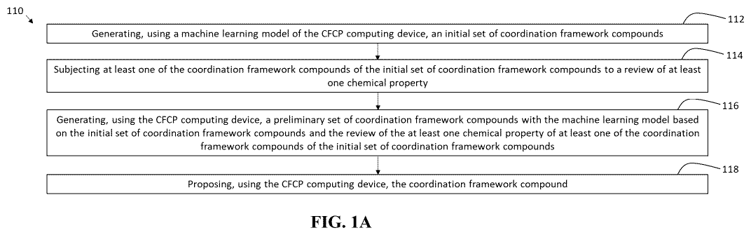

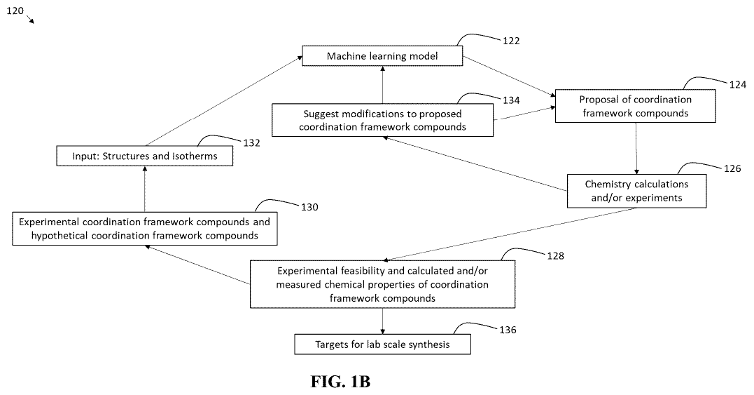

Turning now to the figures, FIG. 1A is an exemplary method flow chart 110. Method flow chart 110 depicts exemplary steps of the method embodiments described herein, and is not intended to limit the method embodiments. In the exemplary embodiment, an initial set of coordination framework compounds is generated 112 using a machine learning model of the CFCP computing device. At least one of the coordination framework compounds of the initial set of coordination framework compounds is subjected 114 to a review of at least one chemical property. A preliminary set of coordination framework compounds is generated 116 with the machine learning model, using the CFCP computing device, based on the initial set of coordination framework compounds and the review of the at least one chemical property of at least one of the coordination framework compounds of the initial set of coordination framework compounds. The coordination framework compound is proposed 118 using the CFCP computing device.

The method flow chart 110 of FIG. 1A may be recursive and/or iterative, such that any of the exemplary steps may be performed more than once and/or used to inform another of the exemplary steps. One such exemplary embodiment is a hybrid ML-Chemistry coordination framework compound suggestion workflow shown in FIG. 1B. In this active learning workflow embodiment, method flow chart 120 depicts exemplary steps of the method embodiments described herein, and is not intended to limit the method embodiments. In the exemplary embodiment, a machine learning model 122 outputs 124 a proposal of coordination framework compounds. At least one of the coordination framework compounds of the proposal of coordination framework compounds is subjected 126 to chemistry calculations and/or experiments. The results of the chemistry calculations and/or experiments may lead to a suggestion 134 of modifications to the proposed coordination framework compounds. This suggestion 134 of modifications can either be directed to the machine learning model 122 or outputted 124 as a proposal of coordination framework compounds. The experimental feasibility and calculated and/or measured chemical properties of coordination framework compounds are determined 128. Proposed compounds that possess desirable properties may be marked 136 as targets for lab scale synthesis. Other proposed compounds may be compared 130 to experimental coordination framework compounds and hypothetical coordination framework compounds. The comparison may further include inputs 132 of structures and isotherms. The learning of this method may be reapplied to the machine learning model until satisfactory results are achieved.

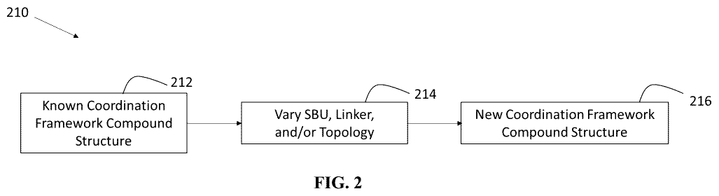

FIG. 2 is an exemplary flow chart 210 for use in proposing new coordination framework compounds based on known coordination framework compounds. Object 212 includes starting from a known coordination framework compound. Object 214 includes varying a SBU, linker, and/or topology of the known coordination framework compound. Object 216 includes proposing the new coordination framework compound structure based on the variations of the known coordination framework compound. This scheme is more targeted and permits immediate suggestion of coordination framework compounds. However, it requires substantial chemical insights.

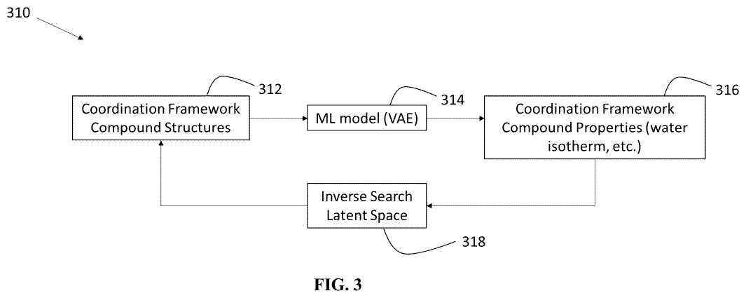

FIG. 3 is an exemplary objective flow chart 310. Object 312 includes inputs of coordination framework compounds. Object 314 includes a ML model, such as VAE, that is updated with the inputs. Object 316 includes a prediction of coordination framework compound properties, such as water isotherms. Object 318 includes an inverse search latent space to suggest and verify new coordination framework compounds. These new coordination framework compounds can be used as inputs in Object 312 in subsequent iterations of exemplary objective flow chart 310. Exemplary objective flow chart 310 allows for learning of coordination framework compound construction information based on known coordination framework compounds. It includes a good latent space that represents the coordination framework compound space. It requires large amounts of coordination framework compound structures with chemical properties such as pore volume, H-bonding site density, and water isotherms. In some embodiments, the model may include large amounts of coordination framework compound structures with DFT/GCMC or DFT/GEMC calculated water isotherms calculated with techniques such as density functional theory (DFT) and/or grand canonical Monte Carlo (GCMC) calculations and/or Gibbs ensemble Monte Carlo (GEMC) calculations. It can explore coordination framework compounds without rod-like metal nodes.

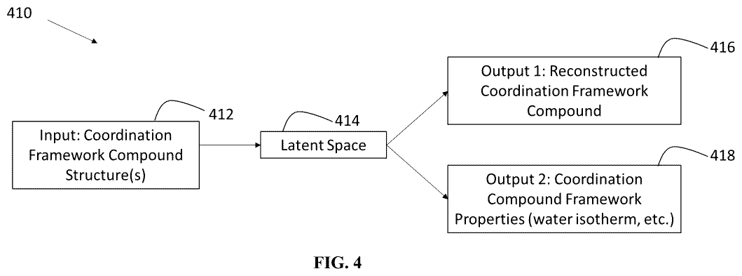

FIG. 4 is an exemplary ML model flow chart 410. Object 412 includes inputs of coordination framework compounds. Object 414 includes a latent space that is low dimensional compared with the input, but enables searching for desired coordination framework compound properties, as well as reconstruction and mapping to new coordination framework compounds. Object 416 includes a first output including a reconstructed coordination framework compound. Object 418 includes a second output including coordination framework compound properties, such as a water isotherm.

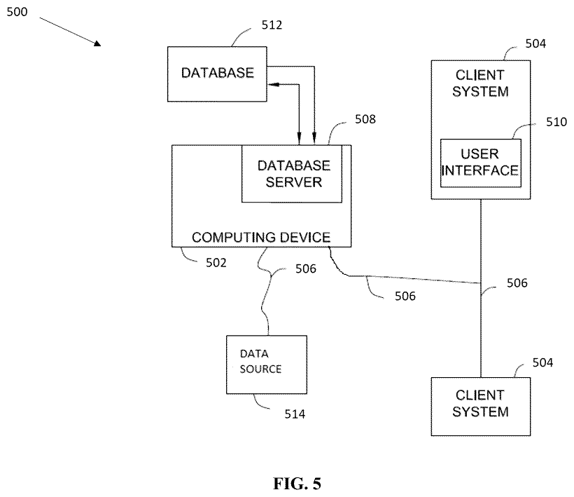

FIG. 5 is a block diagram of an exemplary embodiment of a computer system 500 used in proposing coordination framework compounds that includes a computing device 502 in accordance with one exemplary embodiment of the present disclosure. Computing device 502 may also be referred to herein as a coordination framework compound proposal (CFCP) computing device. In the exemplary embodiment, system 500 is used for proposing coordination framework compounds, as described herein. Computer system 500 may be used to implement one or more of the methods described herein.

More specifically, in the exemplary embodiment, system 500 includes computing device 502, and a plurality of client sub-systems, also referred to as client systems 504, connected to computing device 502. In one embodiment, client systems 504 are computers including a web browser, such that computing device 502 is accessible to client systems 504 using the Internet and/or using network 506. Client systems 504 are interconnected to the Internet through many interfaces including a network 506, such as a local area network (LAN) or a wide area network (WAN), dial-in-connections, cable modems, special high-speed Integrated Services Digital Network (ISDN) lines, and RDT networks. Client systems 504 may include external systems used to store data. Computing device 502 is also in communication with one or more data sources 514 using network 506. Further, client systems 504 may additionally communicate with data sources 514 using network 506. Further, in some embodiments, one or more client systems 504 may serve as data sources 514, as described herein. Client systems 504 may be any device capable of interconnecting to the Internet including a web-based phone, PDA, or other web-based connectable equipment.

A database server 508 is connected to a database 512, which contains information on a variety of matters, as described below in greater detail. In one embodiment, centralized database 512 is stored on device 502 and can be accessed by potential users at one of client systems 504 by logging onto computing device 502 through one of client systems 504. In an alternative embodiment, database 512 is stored remotely from device 502 and may be non-centralized. Database 512 may be a database configured to store information used by computing device 502 including, for example, transaction records, as described herein.

Database 512 may include a single database having separated sections or partitions, or may include multiple databases, each being separate from each other. Database 512 may store data received from data sources 514 and generated by computing device 502. For example, database 512 may store coordination framework compound data, as described in detail herein.

In the exemplary embodiment, client systems 504 may be associated with any party capable of using system 500 as described herein. In the exemplary embodiment, at least one of client systems 504 includes a user interface 510. For example, user interface 510 may include a graphical user interface with interactive functionality, such that coordination framework compound data and proposals, transmitted from computing device 502 to client system 504, may be shown in a graphical format. A user of client system 504 may interact with user interface 510 to view, explore, and otherwise interact with the displayed information.

In the exemplary embodiment, computing device 502 receives data from a plurality of data sources 514, and aggregates and analyzes the received data (e.g., using machine learning) to propose coordination framework compounds, as described in detail herein.

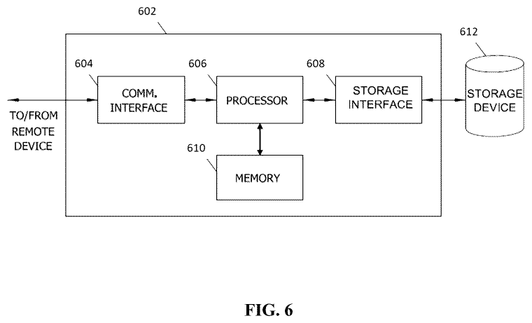

FIG. 6 illustrates an exemplary configuration of a server system 602 such as a computing device, in accordance with one exemplary embodiment of the present disclosure. Server system 602 may be used to implement one or more of the methods described herein. Server system 602 may also include, but is not limited to, a database server (not shown). In the exemplary embodiment, server system 602 proposes coordination framework compounds as described herein.

Server system 602 includes a processor 606 for executing instructions. Instructions may be stored in a memory area 610, for example. Processor 606 may include one or more processing units (e.g., in a multi-core configuration) for executing instructions. The instructions may be executed within a variety of different operating systems on the server system 602, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or other suitable programming languages, etc.).

Processor 606 is operatively coupled to a communication interface 604 such that server system 602 is capable of communicating with a remote device such as a user system or another server system 602. For example, communication interface 604 may receive requests from a client system via the Internet (not shown).

Processor 606 may also be operatively coupled to a storage device 612. Storage device 612 is any computer-operated hardware suitable for storing and/or retrieving data. In some embodiments, storage device 612 is integrated in server system 602. For example, server system 602 may include one or more hard disk drives as storage device 612. In other embodiments, storage device 612 is external to server system 602 and may be accessed by a plurality of server systems 602. For example, storage device 612 may include multiple storage units such as hard disks or solid state disks in a redundant array of inexpensive disks (RAID) configuration. Storage device 612 may include a storage area network (SAN) and/or a network attached storage (NAS) system.

In some embodiments, processor 606 is operatively coupled to storage device 612 via a storage interface 608. Storage interface 608 is any component capable of providing processor 606 with access to storage device 612. Storage interface 608 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 606 with access to storage device 612.

Memory area 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for storage of a computer program.

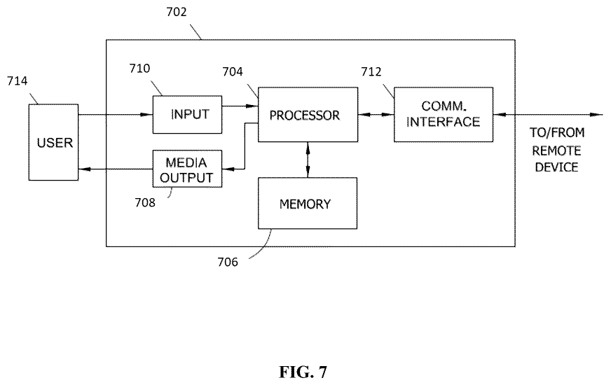

FIG. 7 illustrates an exemplary configuration of a client computing device 702. Client computing device 702 may be used to implement one or more of the methods described herein. Client computing device 702 may include, but is not limited to, client systems (“client computing devices”) 504. Client computing device 702 includes a processor 704 for executing instructions. In some embodiments, executable instructions are stored in a memory area 706. Processor 704 may include one or more processing units (e.g., in a multi-core configuration). Memory area 706 is any device allowing information such as executable instructions and/or other data to be stored and retrieved. Memory area 706 may include one or more computer-readable media.

Client computing device 702 also includes at least one media output component 708 for presenting information to a user 714. Media output component 708 is any component capable of conveying information to user 714. In some embodiments, media output component 708 includes an output adapter such as a video adapter and/or an audio adapter. An output adapter is operatively coupled to processor 704 and operatively couplable to an output device such as a display device (e.g., a liquid crystal display (LCD), organic light emitting diode (OLED) display, cathode ray tube (CRT), or “electronic ink” display) or an audio output device (e.g., a speaker or headphones).

In some embodiments, client computing device 702 includes an input device 710 for receiving input from user 714. Input device 710 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 708 and input device 710.

Client computing device 702 may also include a communication interface 712, which is communicatively couplable to a remote device such as server system 301 or a web server. Communication interface 712 may include, for example, a wired or wireless network adapter or a wireless data transceiver for use with a mobile phone network (e.g., Global System for Mobile communications (GSM), 3G, 4G, 5G, or Bluetooth) or other mobile data network (e.g., Worldwide Interoperability for Microwave Access (WIMAX)).

Stored in memory area 706 are, for example, computer-readable instructions for providing a user interface to user 714 via media output component 708 and, optionally, receiving and processing input from input device 710. A user interface may include, among other possibilities, a web browser and client application. Web browsers enable users 714 to display and interact with media and other information typically embedded on a web page or a website from a web server. A client application allows users 714 to interact with a server application. The user interface, via one or both of a web browser and a client application, facilitates display of information provided by computing device 502. The client application may be capable of operating in both an online mode (in which the client application is in communication with computing device 502) and an offline mode (in which the client application is not in communication with computing device 502).

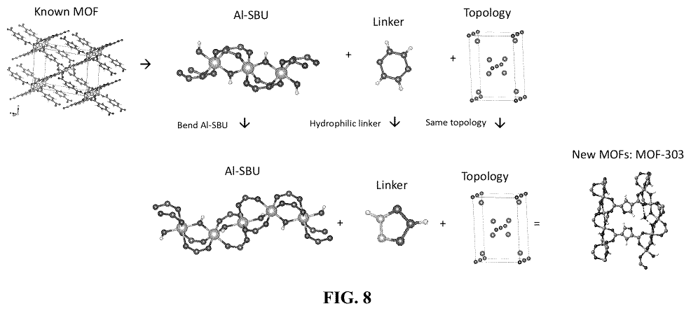

FIG. 8 is an exemplary embodiment depicting the proposal of a new coordination framework compound based on a known coordination framework compounds. It is an example of the method of FIG. 2 and illustrates how chemical insights are incorporated in the present methods. In FIG. 8, a known MOF, MOF 303; Al(OH)(1H-pyrazole-3,5-dicarboxylate), is considered. This known MOF possesses a rod-like SBU with alternating cis-trans corner-shared aluminum octahedra. The SBU is set as a bend aluminum-SBU and the topology is maintained. The linker is varied to produce new MOFs that are variants of the known MOF-303 MOF. In this example, varying any of the SBU, the linker, and the topology will yield a new MOF. This approach is more targeted with immediate MOF suggestion, but requires chemical sense and knowledge. Yet the data requirements may be reduced in this method where one variable is restrained, such as by focusing on MOFs with Al-SBUs.

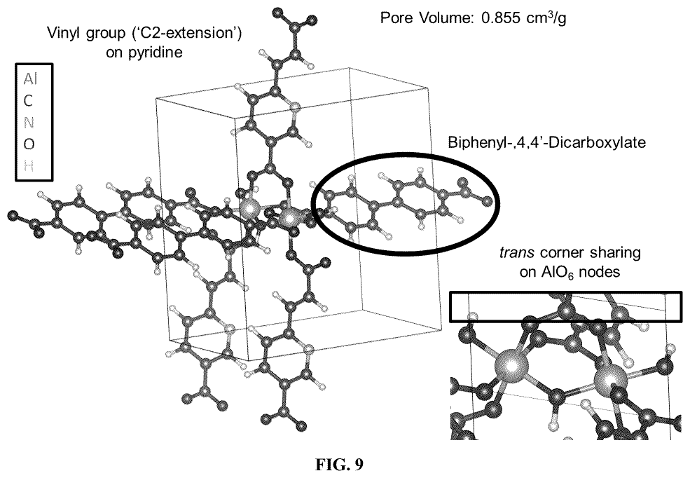

FIG. 9 is an exemplary coordination framework compound, in particular a MOF, obtained from a ML model. The ML model analyzed the structure and composition of the MOF and suggested the use of mixed short-long linkers. This suggestion was subjected to chemical review, where it was determined that replacing the biphenyl-4,4′-dicarboxylate linker with a shorter linker would improve hydrophilicity, and also that replacing the biphenyl-4,4′-dicarboxylate linker with a longer but more hydrophilic linker would increase the pore volume and potentially improve the water isotherms.

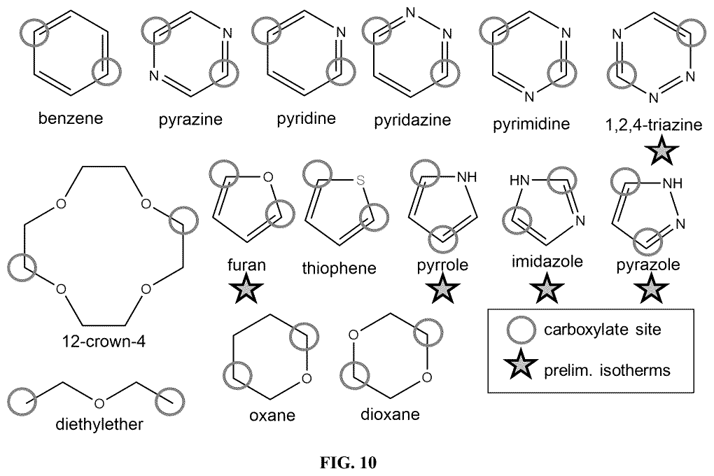

FIG. 10 includes exemplary molecules used in replacement linkers in coordination framework compounds in accordance with the present disclosure. As can be seen, each of the fifteen replacement base molecules has two carboxylate sites. Preliminary isotherms were obtained for five of the replacement base molecules.

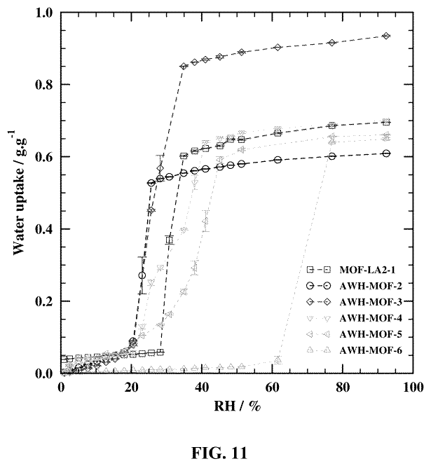

FIG. 11 includes a comparison between properties of comparative coordination framework compounds and one of the coordination framework compounds designed according to the present disclosure. As can be seen, the coordination framework compounds according to the present disclosure exhibited equivalent or significantly improved water uptake.

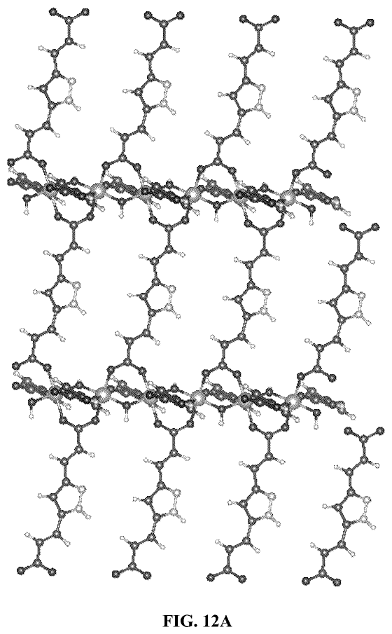

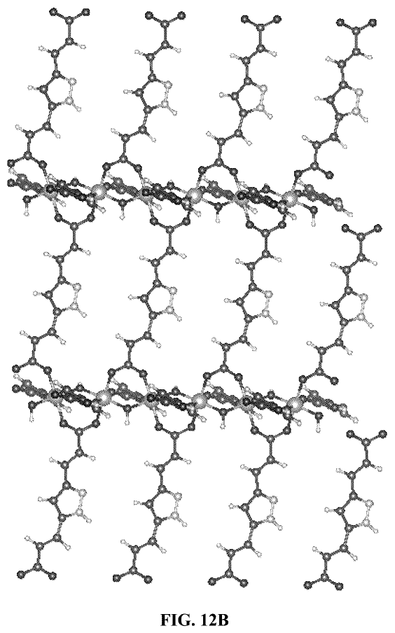

FIGS. 12-16 include exemplary coordination framework compounds proposed by the hybrid ML-Chemistry MOF proposal workflow.

Further aspects of the present disclosure are provided by the subject matter of the following clauses:

1. A method of proposing a coordination framework compound, the method performed using a coordination framework compound proposal (CFCP) computing device that includes a processor coupled to a memory device, the method comprising:
generating, using a machine learning model of the CFCP computing device, an initial set of coordination framework compounds;
subjecting at least one of the coordination framework compounds of the initial set of coordination framework compounds to a review of at least one chemical property;
generating, using the CFCP computing device, a preliminary set of coordination framework compounds with the machine learning model based on the initial set of coordination framework compounds and the review of the at least one chemical property of at least one of the coordination framework compounds of the initial set of coordination framework compounds; and
proposing, using the CFCP computing device, the coordination framework compound.

2. The method in accordance with the preceding clause, further comprising subjecting at least one of the coordination framework compounds of the preliminary set of coordination framework compounds to a review of at least one chemical property.

3. The method in accordance with any preceding clause, further comprising validating at least one of the coordination framework compounds of the preliminary set of coordination framework compounds.

4. The method in accordance with any preceding clause, further comprising performing at least one iteration of a sequence comprising:
generating a further preliminary set of coordination framework compounds with the machine learning model based on at least one generated set of coordination framework compounds and a review of at least one chemical property of at least one of the coordination framework compounds of at least one generated set of coordination framework compounds;
optionally subjecting at least one of the coordination framework compounds of the further preliminary set of coordination framework compounds to a review of at least one chemical property; and
optionally validating at least one of the coordination framework compounds of the further preliminary set of coordination framework compounds.

5. The method in accordance with any preceding clause, wherein the coordination framework compound is a coordination framework compound of the initial set of coordination framework compounds, a coordination framework compound of the preliminary set of coordination framework compounds, or a coordination framework compound of the further preliminary set of coordination framework compounds.

6. The method in accordance with any preceding clause, wherein the coordination framework compound comprises a secondary building unit (SBU), a linker, and a topology.

7. The method in accordance with any preceding clause, wherein the coordination framework compound is a metal organic framework (MOF) compound or a covalent organic framework (COF) compound.

8. The method in accordance with any preceding clause, wherein the machine learning model is trained with existing coordination framework compounds.

9. The method in accordance with any preceding clause, wherein the machine learning model uses at least one technique selected from the group consisting of latent space, inverse search, variational autoencoder (VAE), crystal diffusion variational autoencoder (CDVAE), inverse search of VAE latent space, graph neural network (GNN), neural network, optimization, and combinations thereof.

10. The method in accordance with any preceding clause, wherein the machine learning model is trained to learn a latent space configured to reconstruct coordination framework compound crystal structures and accurately predict associated target properties.

11. The method in accordance with any preceding clause, wherein the at least one chemical property is selected from the group consisting of adsorbate uptake capacity, adsorbate uptake kinetics, adsorbate gravimetric productivity, adsorbate volumetric productivity, adsorbate isotherms and isobars, pore size, pore volume, heat of adsorption, isosteric heat of adsorption, chemical stability, thermal stability, mechanical stability, synthetic feasibility, zeta potential, surface energy, hydrophobicity, hydrophilicity, chemical performance, chemical modifications for improvement, and combinations thereof.

12. The method in accordance with any preceding clause, wherein the machine learning model generates the preliminary coordination framework compound based on at least one input selected from the group consisting of crystal structures of existing coordination framework compounds, target properties, target chemical properties, sorption isotherms, moisture sorption isotherms, pore volume, pore size, water stability, hydrophilicity, and combinations thereof.

13. The method in accordance with any preceding clause, wherein the review of at least one chemical property is performed by a machine, a human, or a combination thereof.

14. A coordination framework compound proposal (CFCP) computing device comprising:
a memory; and
a processor communicatively coupled to the memory, the processor programmed to:
generate an initial set of coordination framework compounds with a machine learning model;
subject at least one of the coordination framework compounds of the initial set of coordination framework compounds to a review of at least one chemical property;
generate a preliminary set of coordination framework compounds with the machine learning model based on the initial set of coordination framework compounds and the review of the at least one chemical property of at least one of the coordination framework compounds of the initial set of coordination framework compounds; and
propose the coordination framework compound.

15. The CFCP computing device in accordance with the preceding clause, wherein the processor is further programmed to subject at least one of the coordination framework compounds of the preliminary set of coordination framework compounds to a review of at least one chemical property.

16. The CFCP computing device in accordance with any preceding clause, wherein the processor is further programmed to validate at least one of the coordination framework compounds of the preliminary set of coordination framework compounds.

17. The CFCP computing device in accordance with any preceding clause, wherein the processor is further programmed to perform at least one iteration of a sequence comprising:
generating a further preliminary set of coordination framework compounds with the machine learning model based on at least one generated set of coordination framework compounds and a review of at least one chemical property of at least one of the coordination framework compounds of at least one generated set of coordination framework compounds;
optionally subjecting at least one of the coordination framework compounds of the further preliminary set of coordination framework compounds to a review of at least one chemical property; and
optionally validating at least one of the coordination framework compounds of the further preliminary set of coordination framework compounds.

18. The CFCP computing device in accordance with any preceding clause, wherein the coordination framework compound is a coordination framework compound of the initial set of coordination framework compounds, a coordination framework compound of the preliminary set of coordination framework compounds, or a coordination framework compound of the further preliminary set of coordination framework compounds.

19. The CFCP computing device in accordance with any preceding clause, wherein the coordination framework compound comprises a secondary building unit (SBU), a linker, and a topology.

20. The CFCP computing device in accordance with any preceding clause, wherein the coordination framework compound is a metal organic framework (MOF) compound or a covalent organic framework (COF) compound.

21. The CFCP computing device in accordance with any preceding clause, wherein the machine learning model is trained with existing coordination framework compounds.

22. The CFCP computing device in accordance with any preceding clause, wherein the machine learning model uses at least one technique selected from the group consisting of latent space, inverse search, variational autoencoder (VAE), crystal diffusion variational autoencoder (CDVAE), inverse search of VAE latent space, graph neural network (GNN), neural network, optimization, and combinations thereof.

23. The CFCP computing device in accordance with any preceding clause, wherein the machine learning model is trained to learn a latent space configured to reconstruct coordination framework compound crystal structures and accurately predict associated target properties.

24. The CFCP computing device in accordance with any preceding clause, wherein the at least one chemical property is selected from the group consisting of adsorbate uptake capacity, adsorbate uptake kinetics, adsorbate gravimetric productivity, adsorbate volumetric productivity, adsorbate isotherms and isobars, pore size, pore volume, heat of adsorption, isosteric heat of adsorption, chemical stability, thermal stability, mechanical stability, synthetic feasibility, zeta potential, surface energy, hydrophobicity, hydrophilicity, chemical performance, chemical modifications for improvement, and combinations thereof.

25. The CFCP computing device in accordance with any preceding clause, wherein the machine learning model generates the preliminary coordination framework compound based on at least one input selected from the group consisting of crystal structures of existing coordination framework compounds, target properties, target chemical properties, sorption isotherms, moisture sorption isotherms, pore volume, pore size, water stability, hydrophilicity, and combinations thereof.

26. The CFCP computing device in accordance with any preceding clause, wherein the review of at least one chemical property is performed by a machine, a human, or a combination thereof.

27. A non-transitory computer-readable storage medium having computer-executable instructions embodied thereon, wherein when executed by a coordination framework compound proposal (CFCP) computing device including at least one processor in communication with a memory, the computer-readable instructions cause the CFCP computing device to:
generate an initial set of coordination framework compounds with a machine learning model;
subject at least one of the coordination framework compounds of the initial set of coordination framework compounds to a review of at least one chemical property;
generate a preliminary set of coordination framework compounds with the machine learning model based on the initial set of coordination framework compounds and the review of the at least one chemical property of at least one of the coordination framework compounds of the initial set of coordination framework compounds; and
propose the coordination framework compound.

28. The non-transitory computer-readable storage medium in accordance with the preceding clause, wherein the computer-readable instructions further cause the CFCP computing device to subject at least one of the coordination framework compounds of the preliminary set of coordination framework compounds to a review of at least one chemical property.

29. The non-transitory computer-readable storage medium in accordance with the preceding clause, wherein the computer-readable instructions further cause the CFCP computing device to validate at least one of the coordination framework compounds of the preliminary set of coordination framework compounds.

30. The non-transitory computer-readable storage medium in accordance with the preceding clause, wherein the computer-readable instructions further cause the CFCP computing device to perform at least one iteration of a sequence comprising:
generating a further preliminary set of coordination framework compounds with the machine learning model based on at least one generated set of coordination framework compounds and a review of at least one chemical property of at least one of the coordination framework compounds of at least one generated set of coordination framework compounds;
optionally subjecting at least one of the coordination framework compounds of the further preliminary set of coordination framework compounds to a review of at least one chemical property; and
optionally validating at least one of the coordination framework compounds of the further preliminary set of coordination framework compounds.

31. The non-transitory computer-readable storage medium in accordance with any preceding clause, wherein the coordination framework compound is a coordination framework compound of the initial set of coordination framework compounds, a coordination framework compound of the preliminary set of coordination framework compounds, or a coordination framework compound of the further preliminary set of coordination framework compounds.

32. The non-transitory computer-readable storage medium in accordance with any preceding clause, wherein the coordination framework compound comprises a secondary building unit (SBU), a linker, and a topology.

33. The non-transitory computer-readable storage medium in accordance with any preceding clause, wherein the coordination framework compound is a metal organic framework (MOF) compound or a covalent organic framework (COF) compound.

34. The non-transitory computer-readable storage medium in accordance with any preceding clause, wherein the machine learning model is trained with existing coordination framework compounds.

35. The non-transitory computer-readable storage medium in accordance with any preceding clause, wherein the machine learning model uses at least one technique selected from the group consisting of latent space, inverse search, variational autoencoder (VAE), crystal diffusion variational autoencoder (CDVAE), inverse search of VAE latent space, graph neural network (GNN), neural network, optimization, and combinations thereof.

36. The non-transitory computer-readable storage medium in accordance with any preceding clause, wherein the machine learning model is trained to learn a latent space configured to reconstruct coordination framework compound crystal structures and accurately predict associated target properties.

37. The non-transitory computer-readable storage medium in accordance with any preceding clause, wherein the at least one chemical property is selected from the group consisting of adsorbate uptake capacity, adsorbate uptake kinetics, adsorbate gravimetric productivity, adsorbate volumetric productivity, adsorbate isotherms and isobars, pore size, pore volume, heat of adsorption, isosteric heat of adsorption, chemical stability, thermal stability, mechanical stability, synthetic feasibility, zeta potential, surface energy, hydrophobicity, hydrophilicity, chemical performance, chemical modifications for improvement, and combinations thereof.

38. The non-transitory computer-readable storage medium in accordance with any preceding clause, wherein the machine learning model generates the preliminary coordination framework compound based on at least one input selected from the group consisting of crystal structures of existing coordination framework compounds, target properties, target chemical properties, sorption isotherms, moisture sorption isotherms, pore volume, pore size, water stability, hydrophilicity, and combinations thereof.

39. The non-transitory computer-readable storage medium in accordance with any preceding clause, wherein the review of at least one chemical property is performed by a machine, a human, or a combination thereof.

40. A coordination framework compound comprising:
a plurality of secondary building units (SBUs);
a plurality of linkers forming linkages between the plurality of SBUs; and
a plurality of pores formed in interstices between the linkages;
wherein the coordination framework compound comprises at least one of:
at least two chemically different linkers;
at least two geometrically different pores; and
at least two linkages between two SBUs of the plurality of SBUs.

41. The coordination framework compound in accordance with the preceding clause, wherein the coordination framework compound is selected from the group consisting of metal organic framework (MOF) compounds, covalent organic framework (COF) compounds, zeolitic imidazolate framework (ZIF) compounds, crystalline porous materials, crystalline open frameworks, reticular chemistry compounds, and combinations thereof.

42. The coordination framework compound in accordance with any preceding clause, wherein at least one SBU of the plurality of SBUs comprises a node comprising an atom selected from the group consisting of:
metal atoms, A₁, or Mg;
B, C, N, O, Si, or P;
transition metal atoms, Fe, Co, Cu, or Zn; and
combinations thereof.

43. The coordination framework compound in accordance with any preceding clause, wherein at least one SBU of the plurality of SBUs comprises a coordination structure selected from the group consisting of polyhedral, tetrahedral, octahedral, cubic, dodecahedral, and combinations thereof.

44. The coordination framework compound in accordance with any preceding clause, wherein the coordination framework compound is planarly symmetrical.

45. The coordination framework compound in accordance with any preceding clause, wherein the coordination framework compound is not planarly symmetrical.

46. The coordination framework compound in accordance with any preceding clause, wherein the at least two chemically different linkers comprise at least two linkers of different lengths.

47. The coordination framework compound in accordance with any preceding clause, wherein the at least two geometrically different pores differ by a geometric property selected from the group consisting of size, shape, and combinations thereof.

48. The coordination framework compound in accordance with any preceding clause, wherein the plurality of linkers comprises a linker selected from the group consisting of:

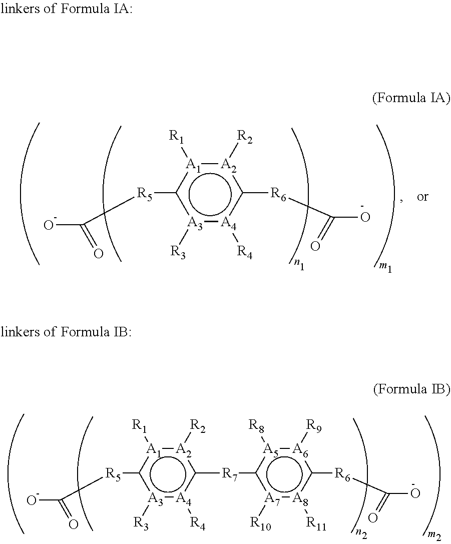

wherein:
n₁, m₁, n₂, and m₂ are each individually selected from the group consisting of 1, 2, 3, 4, 5, 6, 7, 8, 9, 10, integers no more than 100, integers no more than 1000, integers no more than 10000, integers no more than 100000, and integers no more than 1000000;
R₁, R₂, R₃, R₄, R₈, R₉, R₁₀, and R₁₁ are each individually selected from the group consisting of H, NH₂, OH, and SH;
R₅ and R₆ are each individually selected from the group consisting of direct bonds, R₁₂NHR₁₃, R₁₂OR₁₃, R₁₂SR₁₃, C₁-C₆ alkyl optionally substituted with at least one substituent selected from the group consisting of NH₂, OH, and SH, C₁-C₆ alkylene optionally substituted with at least one substituent selected from the group consisting of NH₂, OH, and SH, and combinations thereof;
R₇ is selected from the group consisting of direct bonds, ring fusions, NH, O, S, and C₁-C₆ alkyl;
R₁₂ and R₁₃ are each individually selected from the group consisting of direct bonds, NH, O, S, and C₁-C₆ alkyl; and
A₁, A₂, A₃, A₄, A₅, A₆, A₇, and A₈ are each individually selected from the group consisting of C, N, O, and S;

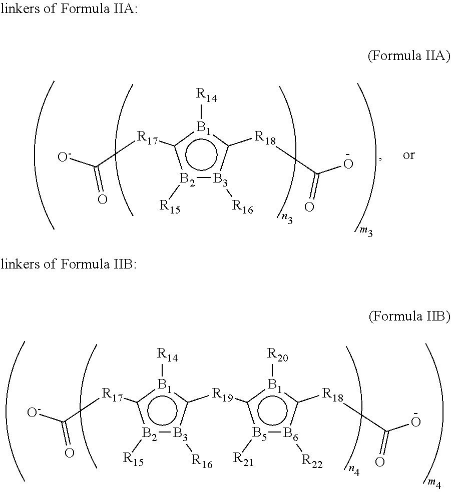

wherein:
n₃, m₃, n₄, and m₄ are each individually selected from the group consisting of 1, 2, 3, 4, 5, 6, 7, 8, 9, 10, integers no more than 100, integers no more than 1000, integers no more than 10000, integers no more than 100000, and integers no more than 1000000;
R₁₄, R₁₅, R₁₆, R₂₀, R₂₁, and R₂₂ are each individually selected from the group consisting of H, NH₂, OH, and SH;
R₁₇ and R₁₈ are each individually selected from the group consisting of direct bonds, R₂₃NHR₂₄, R₂₃OR₂₄, R₂₃SR₂₄, C₁-C₆ alkyl optionally substituted with at least one substituent selected from the group consisting of NH₂, OH, and SH, C₁-C₆ alkylene optionally substituted with at least one substituent selected from the group consisting of NH₂, OH, and SH, and combinations thereof;
R₁₉ is selected from the group consisting of direct bonds, ring fusions, NH, O, S, and C₁-C₆ alkyl;
R₂₃ and R₂₄ are each individually selected from the group consisting of direct bonds, NH, O, S, and C₁-C₆ alkyl; and
B₁, B₂, B₃, B₄, B₅, and B₆ are each individually selected from the group consisting of C, N, O, and S; and
combinations thereof.

49. The coordination framework compound in accordance with any preceding clause, wherein the plurality of linkers comprises a linker selected from the group consisting of:

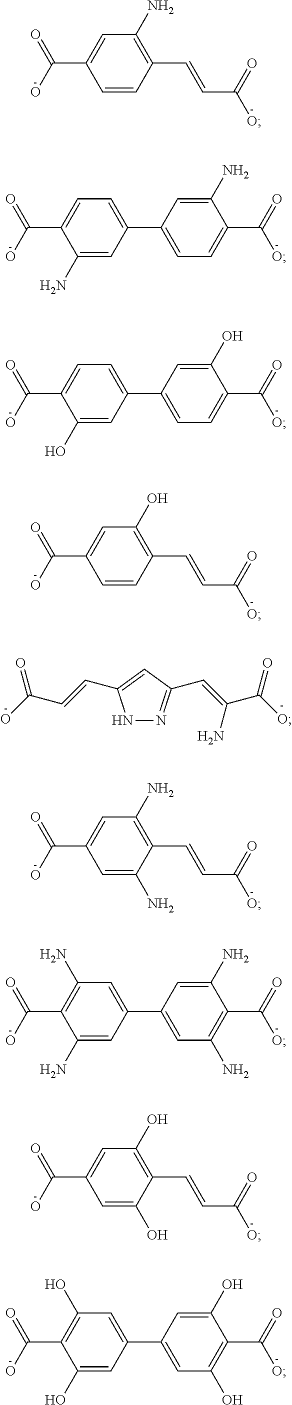

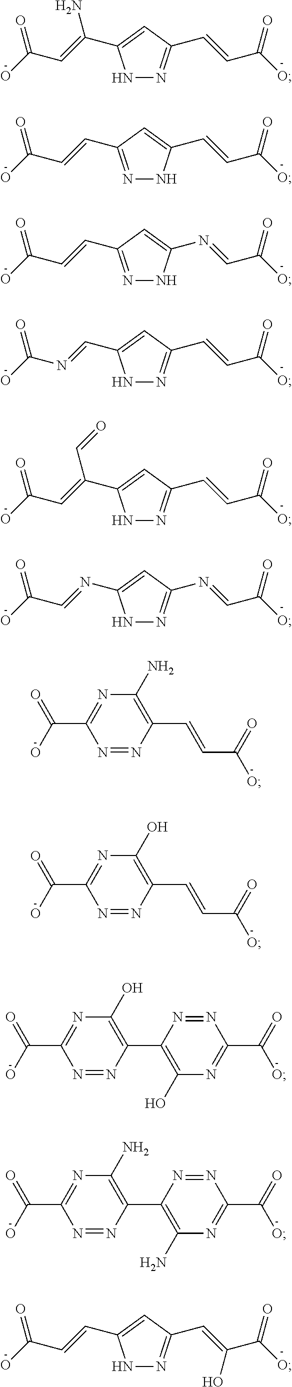

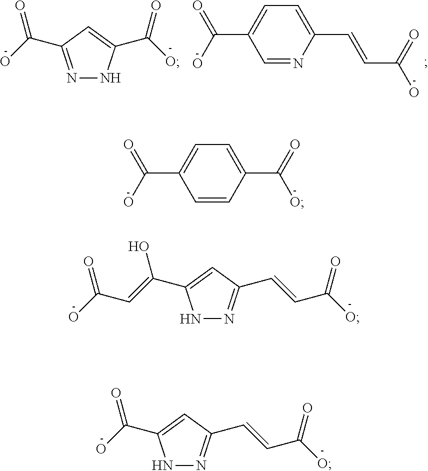

and combinations thereof.

50. The coordination framework compound in accordance with any preceding clause, wherein the plurality of linkers comprises a linker selected from the group consisting of:

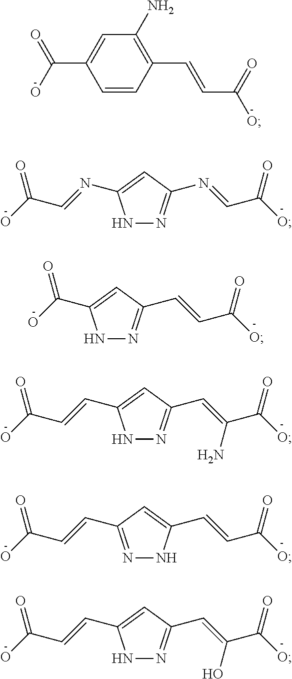

and combinations thereof.

References to “some embodiments” in the above description are not intended to be interpreted as excluding the existence of additional embodiments that also incorporate the recited features.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application. The following Examples can be performed using the CFCP computing device and/or non-transitory computer-readable storage medium described herein.

### Example 1

A hybrid ML-Chemistry MOF proposal workflow was followed as detailed herein.

Initially, a CDVAE model was provided with training data from a MOF database and including MOF-303 variants. MOF structures were optimized, starting from MOF-303 variants, to maximize pore volume (PV). About 10,000 MOFs were generated. After algorithm filtering, there were about 100 filtered MOFs. An exemplary MOF from the ML model is shown in FIG. 9.

Next, the filtered MOFs were subjected to chemical analysis. The CDVAE proposed the use of a mixture of long linker and short linkers. This proposal was further explored by performing Perdew-Burke-Ernzerhof (PBE) density functional theory in a Vienna Ab Initio Simulation Package (VASP) program. A 520 eV energy cutoff was used for the plane waves; soft PAW pseudopotentials were used for the atoms where they were available (H, C, N, O). The k-point mesh included only the I-point. The convergence threshold for the self-consistent field cycles was 106 eV; the force threshold for the geometry optimization was 0.05 e V/Å. There were 39 density functional theory optimized candidates. Different SBUs and different linkers were examined. This chemical analysis revealed that replacing a biphenyl linker with shorter linkers would improve MOF hydrophilicity.

After this chemical analysis, fifteen different compounds optimized by density functional theory were proposed as MOF compounds. These fifteen different compounds included shorter linkers and were chemically evaluated and proposed as MOF compounds. Molecules used in replacement linkers in these fifteen compounds are depicted in FIG. 10. Their corresponding pore volumes are shown below in Table 1. As referenced below, MOF-303 corresponds to Al(OH)(1H-pyrazole-3,5-dicarboxylate). Ultimately, several hypothetical MOFs that have larger pore volume than MOF-303 were identified, and these compounds potentially have improved water isotherms compared to MOF-303.

TABLE 1
Pore volumes for mixed-linker MOFs.

| Parent Molecule for Replacement Linker | MOF Pore Volume |
|---|---|
| (Two Carboxylate Groups Added) | (cm³/g) |
| Benzene | 0.652 |
| Pyrazine | 0.652 |
| Pyridine | 0.652 |
| Pyridazine | 0.650 |
| Pyrimidine | 0.648 |
| 1,2,4-Triazine | 0.637 |
| 12-Crown-4 | 0.579 |
| Diethylether | 0.521 |
| MOF-303 (Experimental Value) | 0.519 |
| Pyrazole | 0.505 |
| Imidazole | 0.504 |
| Pyrrole | 0.502 |
| MOF-303 (PBE-D3(BJ) Value) | 0.498 |
| Furan | 0.496 |
| Thiophene | 0.486 |
| Dioxane | 0.453 |
| Oxane | 0.425 |

### Example 2

Coordination framework compounds proposed by the hybrid ML-Chemistry MOF proposal workflow were simulated with GEMC simulations and their simulated properties were compared to existing coordination framework compounds.

Exemplary coordination framework compounds were simulated according to the below descriptions. For each coordination framework compound, the SBU included cis-trans alternating Al(μ₂-OH rods).

AWE-MOF-2: coordination framework compound including a 50:50 mixture of the following linkers:

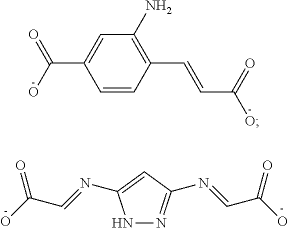

AWE-MOF-3: coordination framework compound including a 50:50 mixture of the following linkers:

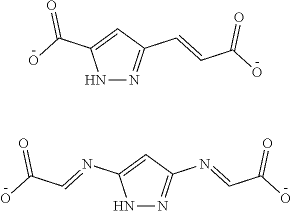 [SMILES: O=C(O)/C=C/c1cc(C(=O)O)[nH]n1 ; O=C(O)/C=N/c1cc(/N=C/C(=O)O)[nH]n1]

AWE-MOF 4: coordination framework compound including a 50:50 mixture of the following linkers:

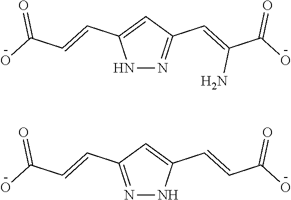 [SMILES: N/C(=C\c1cc(/C=C/C(=O)O)[nH]n1)C(=O)O ; O=C(O)/C=C/c1cc(/C=C/C(=O)O)[nH]n1]

AWE-MOF-5: coordination framework compound including a 50:50 mixture of the following linkers:

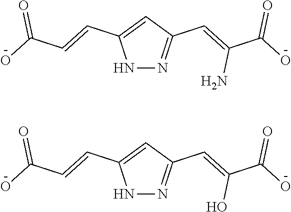 [SMILES: N/C(=C\c1cc(/C=C/C(=O)O)[nH]n1)C(=O)O ; O=C(O)/C=C/c1cc(/C=C(\O)C(=O)O)n[nH]1]

AWE-MOF-6: comparative coordination framework compound including the following linker:

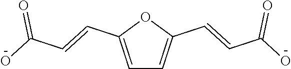 [SMILES: O=C(O)/C=C/c1ccc(/C=C/C(=O)O)o1]

MOF-LA2-1: comparative coordination framework compound including the following linker:

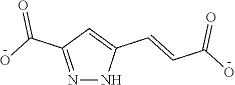 [SMILES: O=C(O)/C=C/c1cc(C(=O)O)n[nH]1]

Results are shown in FIG. 11. Compared to comparative coordination framework compounds, the coordination framework compounds according to the present disclosure exhibited equivalent or significantly improved water uptake.

### Example 3

Coordination framework compounds proposed by the hybrid ML-Chemistry MOF proposal workflow are depicted in FIGS. 12-16.

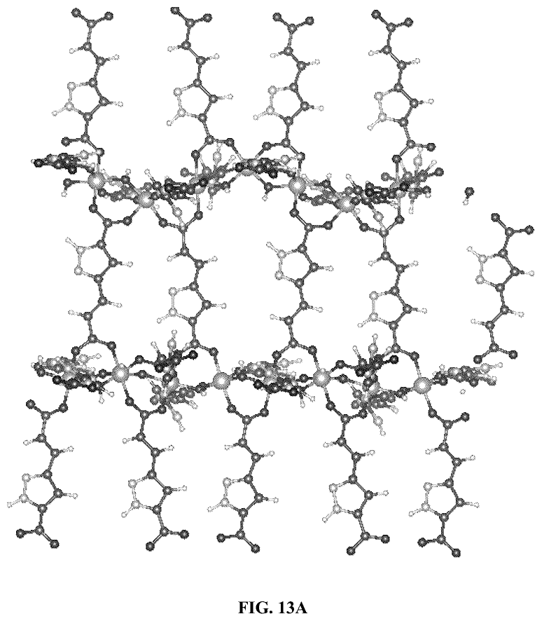

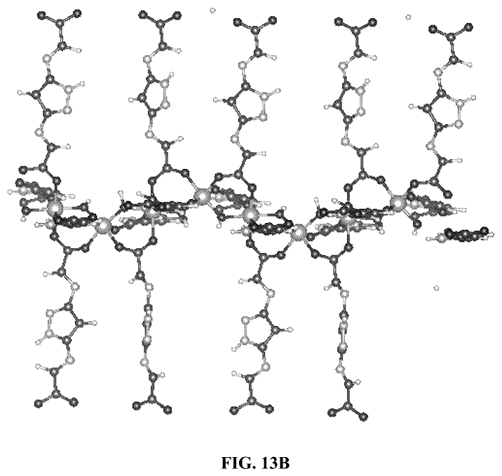

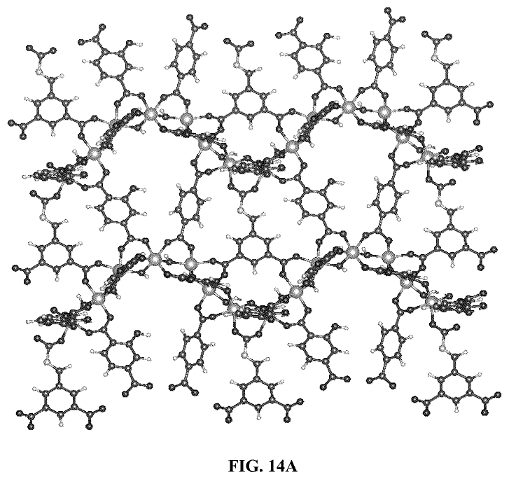

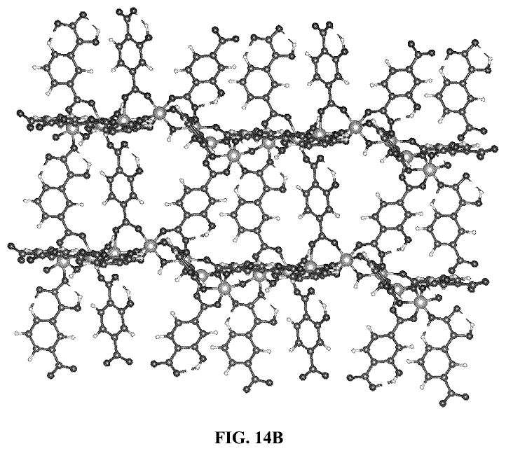

Coordination framework compounds according to the present disclosure may exhibit a variety of topologies. FIGS. 12A-12B depict a coordination framework compound including a straight SBU topology. FIGS. 13A-13B depicts a coordination framework compound including a curly SBU topology. FIGS. 14A-14B depicts a coordination framework compound including an SBU topology that is neither straight nor curly. It is observed that one linker can connect to more than two connection points on SBUs.

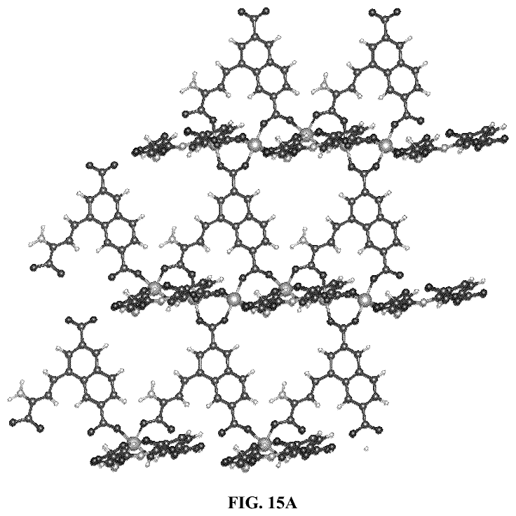

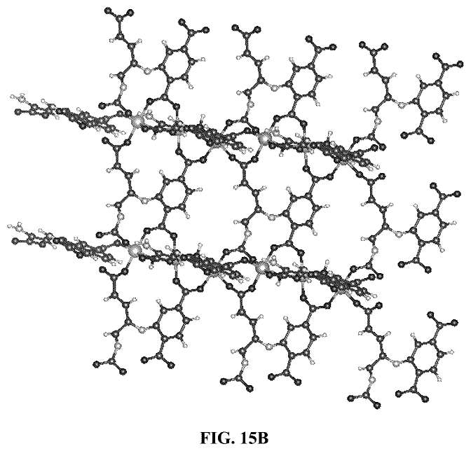

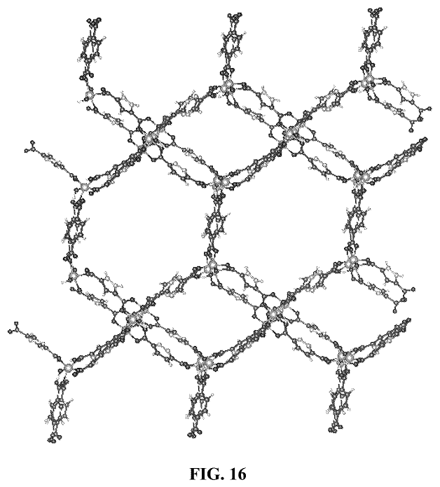

Coordination framework compounds according to the present disclosure may exhibit a variety of linkers and linkages. FIG. 15 depicts a coordination framework compound including a bridging arm linker structure. FIG. 16 depicts a coordination framework compound including a changed pore shape as a result of a mixture of different linkers.

### Conclusions.

Developed herein is a process that combines the strengths of machine learning-based and chemical knowledge-based methods in order to more effectively propose and discover coordination framework compounds. This process is broadly applicable to coordination framework compounds and exemplary coordination framework compounds are demonstrated.

Unless otherwise indicated, approximating language, such as “generally,” “substantially,” and “about,” as used herein indicates that the term so modified may apply to only an approximate degree, as would be recognized by one of ordinary skill in the art, rather than to an absolute or perfect degree. Accordingly, a value modified by a term or terms such as “about,” “approximately,” and “substantially” is not to be limited to the precise value specified. In at least some instances, the approximating language may correspond to the precision of an instrument for measuring the value. Additionally, unless otherwise indicated, the terms “first,” “second,” etc. are used herein merely as labels, and are not intended to impose ordinal, positional, or hierarchical requirements on the items to which these terms refer. Moreover, reference to, for example, a “second” item does not require or preclude the existence of, for example, a “first” or lower-numbered item or a “third” or higher-numbered item.

Although specific features of various embodiments of the invention may be shown in some drawings and not in others, this is for convenience only. Moreover, references to “some embodiments” in the above description are not intended to be interpreted as excluding the existence of additional embodiments that also incorporate the recited features. In accordance with the principles of the invention, any feature of a drawing may be referenced and/or claimed in combination with any feature of any other drawing.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of proposing a coordination framework compound, the method performed using a coordination framework compound proposal (CFCP) computing device that includes a processor coupled to a memory device, the method comprising:
generating, using a machine learning model of the CFCP computing device, an initial set of coordination framework compounds;
subjecting at least one of the coordination framework compounds of the initial set of coordination framework compounds to a review of at least one chemical property;
generating, using the CFCP computing device, a preliminary set of coordination framework compounds with the machine learning model based on the initial set of coordination framework compounds and the review of the at least one chemical property of at least one of the coordination framework compounds of the initial set of coordination framework compounds; and
proposing, using the CFCP computing device, the coordination framework compound.

2. The method of claim 1, further comprising subjecting at least one of the coordination framework compounds of the preliminary set of coordination framework compounds to a review of at least one chemical property.

3. The method of claim 1, further comprising validating at least one of the coordination framework compounds of the preliminary set of coordination framework compounds.

4. The method of claim 1, further comprising performing at least one iteration of a sequence comprising:
generating a further preliminary set of coordination framework compounds with the machine learning model based on at least one generated set of coordination framework compounds and a review of at least one chemical property of at least one of the coordination framework compounds of at least one generated set of coordination framework compounds;
optionally subjecting at least one of the coordination framework compounds of the further preliminary set of coordination framework compounds to a review of at least one chemical property; and
optionally validating at least one of the coordination framework compounds of the further preliminary set of coordination framework compounds.

5. The method of claim 1, wherein the coordination framework compound comprises a secondary building unit (SBU), a linker, and a topology.

6. The method of claim 1, wherein the coordination framework compound is a metal organic framework (MOF) compound or a covalent organic framework (COF) compound.

7. The method of claim 1, wherein the machine learning model is trained with existing coordination framework compounds.

8. The method of claim 1, wherein the machine learning model uses at least one technique selected from the group consisting of latent space, inverse search, variational autoencoder (VAE), crystal diffusion variational autoencoder (CDVAE), inverse search of VAE latent space, graph neural network (GNN), neural network, optimization, and combinations thereof.

9. The method of claim 1, wherein the machine learning model is trained to learn a latent space configured to reconstruct coordination framework compound crystal structures and accurately predict associated target properties.

10. The method of claim 1, wherein the at least one chemical property is selected from the group consisting of adsorbate uptake capacity, adsorbate uptake kinetics, adsorbate gravimetric productivity, adsorbate volumetric productivity, adsorbate isotherms and isobars, pore size, pore volume, heat of adsorption, isosteric heat of adsorption, chemical stability, thermal stability, mechanical stability, synthetic feasibility, zeta potential, surface energy, hydrophobicity, hydrophilicity, chemical performance, chemical modifications for improvement, and combinations thereof.

11. The method of claim 1, wherein the machine learning model generates the preliminary coordination framework compound based on at least one input selected from the group consisting of crystal structures of existing coordination framework compounds, target properties, target chemical properties, sorption isotherms, moisture sorption isotherms, pore volume, pore size, water stability, hydrophilicity, and combinations thereof.

12. The method of claim 1, wherein the review of at least one chemical property is performed by a machine, a human, or a combination thereof.

13. A coordination framework compound proposal (CFCP) computing device comprising:
a memory; and
a processor communicatively coupled to the memory, the processor programmed to:
generate an initial set of coordination framework compounds with a machine learning model;
subject at least one of the coordination framework compounds of the initial set of coordination framework compounds to a review of at least one chemical property;
generate a preliminary set of coordination framework compounds with the machine learning model based on the initial set of coordination framework compounds and the review of the at least one chemical property of at least one of the coordination framework compounds of the initial set of coordination framework compounds; and
propose the coordination framework compound.

14. The CFCP computing device of claim 13, wherein the processor is further programmed to subject at least one of the coordination framework compounds of the preliminary set of coordination framework compounds to a review of at least one chemical property.

15. The CFCP computing device of claim 13, wherein the processor is further programmed to validate at least one of the coordination framework compounds of the preliminary set of coordination framework compounds.

16. The CFCP computing device of claim 13, wherein the processor is further programmed to perform at least one iteration of a sequence comprising:
generating a further preliminary set of coordination framework compounds with the machine learning model based on at least one generated set of coordination framework compounds and a review of at least one chemical property of at least one of the coordination framework compounds of at least one generated set of coordination framework compounds;
optionally subjecting at least one of the coordination framework compounds of the further preliminary set of coordination framework compounds to a review of at least one chemical property; and
optionally validating at least one of the coordination framework compounds of the further preliminary set of coordination framework compounds.

17. A non-transitory computer-readable storage medium having computer-executable instructions embodied thereon, wherein when executed by a coordination framework compound proposal (CFCP) computing device including at least one processor in communication with a memory, the computer-readable instructions cause the CFCP computing device to:
generate an initial set of coordination framework compounds with a machine learning model;
subject at least one of the coordination framework compounds of the initial set of coordination framework compounds to a review of at least one chemical property;
generate a preliminary set of coordination framework compounds with the machine learning model based on the initial set of coordination framework compounds and the review of the at least one chemical property of at least one of the coordination framework compounds of the initial set of coordination framework compounds; and
propose the coordination framework compound.

18. The non-transitory computer-readable storage medium of claim 17, wherein the computer-readable instructions further cause the CFCP computing device to subject at least one of the coordination framework compounds of the preliminary set of coordination framework compounds to a review of at least one chemical property.

19. The non-transitory computer-readable storage medium of claim 17, wherein the computer-readable instructions further cause the CFCP computing device to validate at least one of the coordination framework compounds of the preliminary set of coordination framework compounds.

20. The non-transitory computer-readable storage medium of claim 17, wherein the computer-readable instructions further cause the CFCP computing device to perform at least one iteration of a sequence comprising:
generating a further preliminary set of coordination framework compounds with the machine learning model based on at least one generated set of coordination framework compounds and a review of at least one chemical property of at least one of the coordination framework compounds of at least one generated set of coordination framework compounds;
optionally subjecting at least one of the coordination framework compounds of the further preliminary set of coordination framework compounds to a review of at least one chemical property; and
optionally validating at least one of the coordination framework compounds of the further preliminary set of coordination framework compounds.

21. A coordination framework compound comprising:
a plurality of secondary building units (SBUs);
a plurality of linkers forming linkages between the plurality of SBUs; and
a plurality of pores formed in interstices between the linkages;
wherein the coordination framework compound comprises at least one of:
at least two chemically different linkers;
at least two geometrically different pores; and
at least two linkages between two SBUs of the plurality of SBUs.

22. The coordination framework compound of claim 21, wherein the coordination framework compound is selected from the group consisting of metal organic framework (MOF) compounds, covalent organic framework (COF) compounds, zeolitic imidazolate framework (ZIF) compounds, crystalline porous materials, crystalline open frameworks, reticular chemistry compounds, and combinations thereof.

23. The coordination framework compound of claim 21, wherein at least one SBU of the plurality of SBUs comprises a node comprising an atom selected from the group consisting of:
metal atoms, A₁, or Mg;
B, C, N, O, Si, or P;
transition metal atoms, Fe, Co, Cu, or Zn; and
combinations thereof.

24. The coordination framework compound of claim 21, wherein at least one SBU of the plurality of SBUs comprises a coordination structure selected from the group consisting of polyhedral, tetrahedral, octahedral, cubic, dodecahedral, and combinations thereof.

25. The coordination framework compound of claim 21, wherein the coordination framework compound is planarly symmetrical.

26. The coordination framework compound of claim 21, wherein the coordination framework compound is not planarly symmetrical.

27. The coordination framework compound of claim 21, wherein the at least two chemically different linkers comprise at least two linkers of different lengths.

28. The coordination framework compound of claim 21, wherein the at least two geometrically different pores differ by a geometric property selected from the group consisting of size, shape, and combinations thereof.

29. The coordination framework compound of claim 21, wherein the plurality of linkers comprises a linker selected from the group consisting of:
linkers of Formula IA: 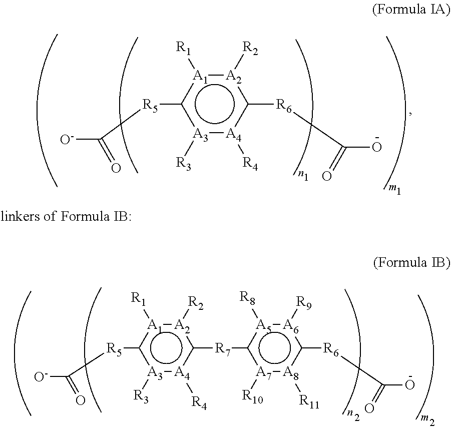
wherein:
n₁, m₁, n₂, and m₂ are each individually selected from the group consisting of 1, 2, 3, 4, 5, 6, 7, 8, 9, 10, integers no more than 100, integers no more than 1000, integers no more than 10000, integers no more than 100000, and integers no more than 1000000;
R₁, R₂, R₃, R₄, R₅, R₉, R₁₀, and R₁₁ are each individually selected from the group consisting of H, NH₂, OH, and SH;
R₅ and R₆ are each individually selected from the group consisting of direct bonds, R₁₂NHR₁₃, R₁₂OR₁₃, R₁₂SR₁₃, C₁-C₆ alkyl optionally substituted with at least one substituent selected from the group consisting of NH₂, OH, and SH, C₁-C₆ alkylene optionally substituted with at least one substituent selected from the group consisting of NH₂, OH, and SH, and combinations thereof;
R₇ is selected from the group consisting of direct bonds, ring fusions, NH, O, S, and C₁-C₆ alkyl;
R₁₂ and R₁₃ are each individually selected from the group consisting of direct bonds, NH, O, S, and C₁-C₆ alkyl; and
A₁, A₂, A₃, A₄, A₅, A₆, A₇, and A₈ are each individually selected from the group consisting of C, N, O, and S; 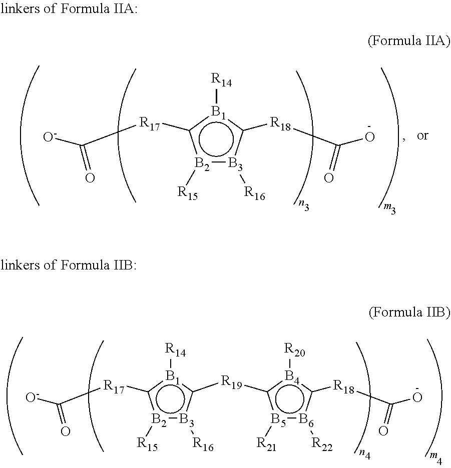
wherein:
n₃, m₃, n₄, and m₄ are each individually selected from the group consisting of 1, 2, 3, 4, 5, 6, 7, 8, 9, 10, integers no more than 100, integers no more than 1000, integers no more than 10000, integers no more than 100000, and integers no more than 1000000;
R₁₄, R₁₅, R₁₆, R₂₀, R₂₁, and R₂₂ are each individually selected from the group consisting of H, NH₂, OH, and SH;
R₁₇ and R₁₈ are each individually selected from the group consisting of direct bonds, R₂₃NHR₂₄, R₂₃OR₂₄, R₂₃SR₂₄, C₁-C₆ alkyl optionally substituted with at least one substituent selected from the group consisting of NH₂, OH, and SH, C₁-C₆ alkylene optionally substituted with at least one substituent selected from the group consisting of NH₂, OH, and SH, and combinations thereof;
R₁₉ is selected from the group consisting of direct bonds, ring fusions, NH, O, S, and C₁-C₆ alkyl;
R₂₃ and R₂₄ are each individually selected from the group consisting of direct bonds, NH, O, S, and C₁-C₆ alkyl; and
B₁, B₂, B₃, B₄, B₅, and B₆ are each individually selected from the group consisting of C, N, O, and S; and
combinations thereof.

30. The coordination framework compound of claim 21, wherein the plurality of linkers comprises a linker selected from the group consisting of: 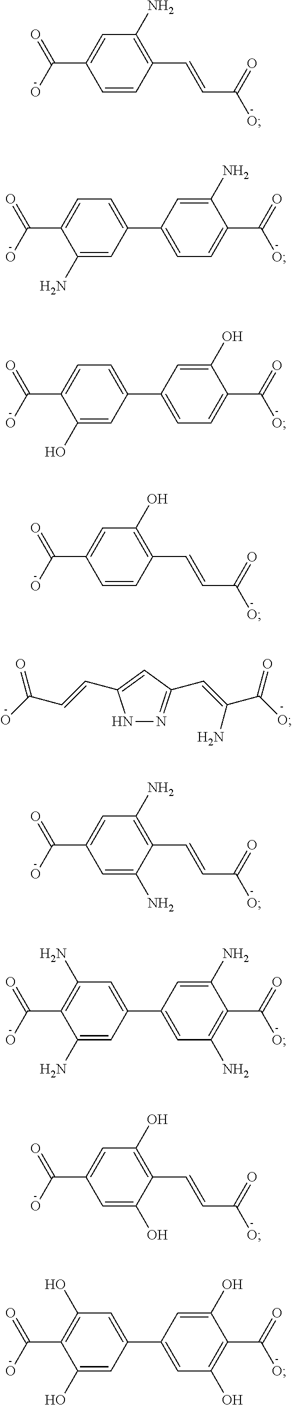 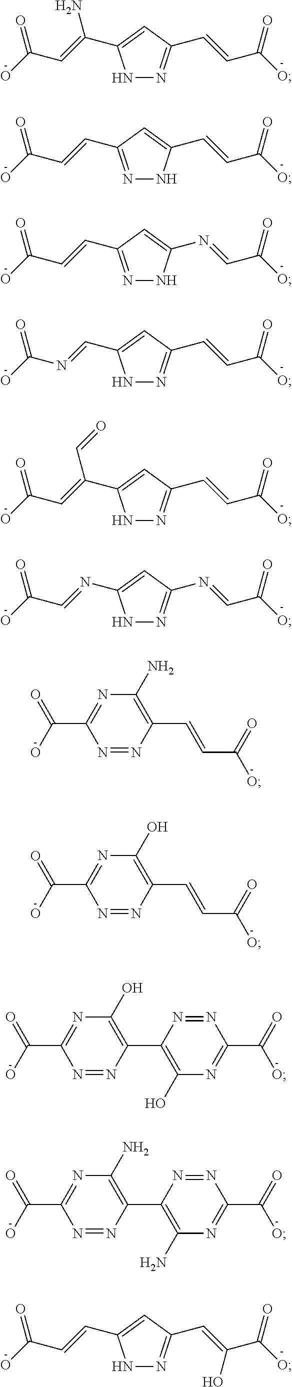 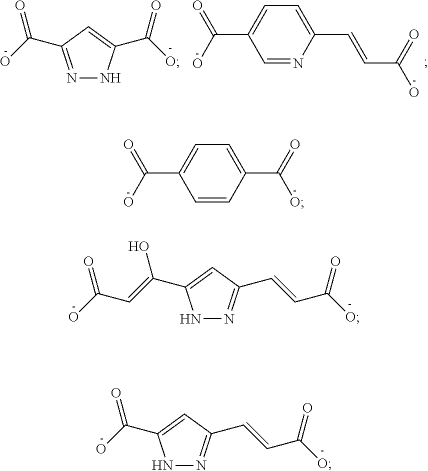
and combinations thereof.

31. The coordination framework compound of claim 21, wherein the plurality of linkers comprises a linker selected from the group consisting of: 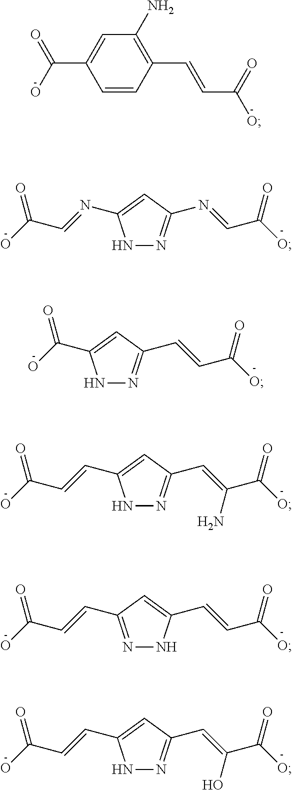
and combinations thereof.

32. A sorbent system including the coordination framework compound of claim 21.
